(19) United States
(12) Patent Application Publication
(10) Pub. No.: US 2003/0176461 A1
(43) Pub. Date: Sep. 18, 2003

(54) Aminopiperidines

(76) Inventors: Ian R. Egle, Ontario (CA); Jennifer Frey, Ontario (CA); Methvin B. Isaac, Ontario (CA); Abdelmalik Slass, Ontario (CA); Leah E. Begleiter, Ontario (CA); Louise G. Edwards, Ontario (CA); Tomislav Stefanac, Ontario (CA); Ashok Tehim, Ridgewood, NJ (US); Shewn P. Maddaford, Ontario (CA); Hoi Lun Allan Tse, Markham (CA)

Correspondence Address: BACON & THOMAS, PLLC, 625 SLATERS LANE, FOURTH FLOOR, ALEXANDRIA, VA, 22314

(21) Appl. No.: 10/257,402

(22) PCT Filed: Apr. 20, 2001
(86) PCT No.: PCT/CA01/00548

(30) Foreign Application Priority Data
Apr. 20, 2000 (US) 60198800

(51) Int. Cl. A61K 31/454; A61K 31/453; C 07D 4 9/02; C 07D 4 1/02; C07D 211/06; A61K 31/445

(52) U.S. Cl. 514/317; 514/326; 514/321; 546/198; 546/207; 546/212; 546/229; 546/206; 514/319

(57) ABSTRACT

Described herein are compounds having general formula (1), wherein groups R₁, R₂, R₃, Ar₁, Ar₂, Ar₃, X, Y, p and n are as defined in the specification, and a salt, solvate and hydrate thereof. Such compounds inhibit glycine transport (or reuptake) via the GlyT-1 transporter, or are precursors (for example, pro-drugs) of such compounds and, thus, are useful in the treatment of schizophrenia, as well as other CNS-related disorders such as dementia, Alzheimer's disease, attention deficit disorder and depression.

The present invention relates to a class of aminopiperidines, to pharmaceutical compositions containing them and to methods of treating neurological and neuropsychiatric disorders using such compounds.

### BACKGROUND OF THE INVENTION

Synaptic transmission is a complex form of intercellular communication that involves a considerable array of specialized structures in both the pre- and post-synaptic terminal and surrounding glial cells (Kanner and Schuldiner, CRC Critical Reviews in Biochemistry, 22, 1987:1032). Transporters sequester neurotransmitter from the synapse, thereby regulating the concentration of neurotransmifter in the synapse, as well as its duration therein, which together influence the magnitude of synaptic transmission. Further, by preventing the spread of transmitter to neighbouring synapses, transporters maintain the fidelity of synaptic transmission. Lastly, by sequestering released transmitter into the presynaptic terminal, transporters allow for neurotransmitter reutilization.

Neurotransmitter transport is dependent upon extracellular sodium and the voltage difference across the membrane. Under conditions of intense neuronal firing, for example, during a seizure, transporters can function in reverse, releasing neurotransmifter in a calcium-independent non-exocytotic manner (Attwell et al., Neuron, 11, 1993:401-407). Pharmacologic modulation of neurotransmitter transporters thus provides a means for modifying synaptic activity, which provides useful therapy for the treatment of neurological and psychiatric disturbances.

The amino acid glycine is a major neurotransmitter in the mammalian central nervous system, functioning at both inhibitory and excitatory synapses. By nervous system, both the central and peripheral portions of the nervous system are intended. These distinct functions of glycine are mediated by two different types of receptor, each of which is associated with a different class of glycine transporter. The inhibitory actions of glycine are mediated by glycine receptors that are sensitive to the convulsant alkaloid strychnine, and are thus referred to as “strychnine-sensitive”. Such receptors contain an intrinsic chloride channel that is opened upon binding of glycine to the receptor; by increasing chloride conductance, the threshold for firing of an action potential is increased. Strychnine-sensitive glycine receptors are tound predominantly in the spinal cord and brainstem, and pharmacological agents that enhance the activation of such receptors will thus increase inhibitory neurotransmission in these regions.

Glycine also functions in excitatory transmission by modulating the actions of glutamate, the major excitatory neurotransmitter in the central nervous system (Johnson and Ascher, Nature, 325, 1987:529-531; Fletcher et al., Glycine Transmission, Otterson and Storm-Mathisen, eds., 1990:193-219). Specifically, glycine is an obligatory co-agonist at the class of glutamate receptor termed N-methyl-D-aspartate (NMDA) receptor. Activation of NMDA receptors increases sodium and calcium conductance, which depolarizes the neuron, thereby increasing the likelihood that it will fire an action potential.

NMDA receptors in the hippocampal region of the brain play an important role in a model of synaptic plasticity known as long-term potentiation (LTP), which is integral in certain types of learning and memory (Hebb, D. O (1949) The Organization of Behavior, Wiley, NY; Bliss and Collingridge (1993) Nature 361: 31-39; Morris et al. (1986) Nature 319: 774-776). Enhanced expression of selected NMDA receptor sub-units in transgenic mice results in increased NMDA-receptor-mediated currents, enhanced LTP, and better performance in some tests of learning and memory (Tang et al. (1999) Nature 401: 63).

Conversely, decreased expression of selected NMDA receptor sub-units in transgenic mice produces behaviors similar to pharmacologically-induced animal models of schizophrenia, including increased locomotion, increased stereotypy, and deficits in social/sexual interactions (Mohn et al. (1999) Cell 98:427-436). These aberrant behaviors can be ameliorated using the antipsychotics haloperidol and clozapine.

NMDA receptors are widely distributed throughout the brain, with a particularly high density in the cerebral cortex and hippocampal formation.

Molecular cloning has revealed the existence of two classes of glycine transporters in mammalian brains, termed GlyT-1 and GlyT-2. GlyT-1 is found throughout the brain and spinal cord, and it has been suggested that its distribution corresponds to that of glutamatergic pathways and NMDA receptors (Smith, et al., Neuron, 8, 1992:927-935). Molecular cloning has further revealed the existence of three variants of GlyT-1, termed GlyT-1a, GlyT-1b, GlyT-1c and GlyT-1d. Two of these variants (1a and 1b) are found in rodents, each of which displays a unique distribution in the brain and peripheral tissues (Borowsky et al., Neuron, 10, 1993:851-863; Adams et al., J. Neuroscience, 15, 1995:2524-2532). The third variant, 1c, has only been detected in human tissues (Kim, et al., Molecular Pharmacology, 45, 1994:608-617). The fourth variant has been detected in human tissue (see U.S. Pat. No. 6,008,015). These variants arise by differential splicing and exon usage, and differ in their N-terminal regions. GlyT-2, in contrast, is found predominantly in the brain stem and spinal cord, and its distribution corresponds closely to that of strychnine-sensitive glycine receptors (Liu et al., J. Biological Chemistry, 268, 1993:22802-22808; Jursky and Nelson, J. Neurochemistry, 64, 1995:1026-1033). Another distinguishing feature of glycine transport mediated by GlyT-2 is that it is not inhibited by sarcosine as is the case for glycine transport mediated by GlyT-1. These data are consistent with the view that, by regulating the synaptic levels of glycine, GlyT-1 and GlyT-2 selectively influence the activity of NMDA receptors and strychnine-sensitive glycine receptors, respectively.

Compounds which inhibit or activate glycine transporters would thus be expected to alter receptor function by modifying glycine concentrations in the synapse and, thus, provide therapeutic benefits in a variety of disease states.

For example, compounds which inhibit GlyT-1 mediated glycine transport may increase glycine concentrations at NMDA receptors, which receptors are located in the forebrain, among other locations. This concentration increase could perhaps elevate the activity of NMDA receptors, thereby possibly alleviating symptoms of schizophrenia and enhancing cognitive function. Alternatively, compounds that interact directly with the glycine receptor component of the NMDA receptor can have the same or similar effects as increasing or decreasing the availability of extracellular glycine caused by inhibiting or enhancing GlyT-1 activity, respectively. See, for example, Pitkänen et al., Eur. J. Pharmacol., 253, 125-129 (1994); Thiels et al., Neuroscience, 46, 501-509 (1992); and Kretschmer and Schmidt, J. Neurosci., 16, 1561-1569 (1996).

### SUMMARY OF THE INVENTION

According to one aspect of the invention, there are provided compounds of Formula l:

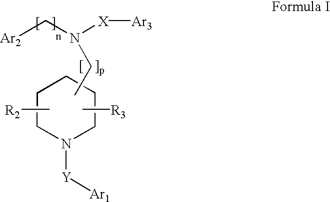

wherein:

X is a C=O group or an SO₂ group;

Y is selected from the group consisting of —[CHR₁]ₘ-, —CH₂CH=CH-, and CH₂C≡C-;

m is an integer selected from the group consisting of 1, 2, and 3;

n is an integer selected from the group consisting of 0, 1, and 2;

p is an integer selected from the group consisting of 0, 1, and 2;

R₁, R₂ and R₃ are independently selected from the group consisting of H and alkyl;

Ar₁, Ar₂ and Ar₃ are independently selected aryl groups, optionally substituted with up to five substituents independently selected from the group consisting of alkyl, alkoxy, cycloalkyl, cycloalkyloxy, heterocycloalkyl, heterocycloalkyloxy, alkanoyl, thioalkyl, aralkyl, aralkoxy, aryloxyalkyl, aryloxyalkoxy, cycloalkyl-substituted alkyl, cycloalkyloxy-substituted alkyl, cycloalkyl-substituted alkoxy, cycloalkyloxy-substituted alkoxy, heterocycloalkyl-substituted alkyl, heterocycloalkyloxy-substituted alkyl heterocycloalkyl-substituted alkoxy, heterocycloalkyloxy-substituted alkoxy, thioaryl, aralkylthio, thioaryl-alky, halo, NO₂, Ph, CF₃, OCF₃, CN, OH, methylenedioxy, ethylenedioxy, SO₂NRR′, NRR′, CO₂R (where R and R′ are independently selected from the group consisting of H and alkyl) and aryl. The aryl substituent may be further substituted with any substituent selected from the preceding list;

with the following provisos:

(i) when X is a C=O group, n is 0 and Ar₂ is not a 2,3-dihydro-indol-5yl or 2,3-dihydro-indol-6-yl group;

(ii) when X is an SO₂ group and n is 0, Ar₂ is not a 2,3-dihydro-indol-5-yl or 2,3-dihydro-indol-6-yl group;

and a salt, solvate and hydrate thereof.

It has been found that compounds of Formula I inhibit glycine transport (or reuptake) via the GlyT-1 transporter, or are precursors (for example, pro-drugs) of such compounds and, thus, are useful in the treatment of schizophrenia, as well as other CNS-related disorders such as dementia Alzheimer's disease, attention deficit disorder and depression.

According to another aspect of the invention, there is provided a pharmaceutical composition comprising a compound of Formula I in an amount effective to inhibit glycine transport, and a pharmaceutically acceptable carrier.

In another aspect of the invention there are provided compositions containing the present compounds in amounts for pharmaceutical use to treat medical conditions for which a glycine transport inhibitor is indicated. Preferred are those compositions containing compounds useful in the treatment of medical conditions for which GlyT-1-mediated inhibition of glycine transport is needed, such as the treatment of schizophrenia or cognitive dysfunction.

Definitions

The term aryl as used herein means a monocyclic aromatic group such as phenyl, pyridyl, furyl, thienyl, and the like, or a benzo-fused aromatic group such as naphthyl, indanyl, quinolinyl, fluorenyl and the like.

The term aryloxy as used herein means an oxygen radical substituted by an aryl group and includes phenoxy and the like

The term alkyl as used herein means straight and branched carbon chain radicals containing 1, 2, 3, 4, 5 or 6 carbon atoms and includes methyl, ethyl and the like.

The term cycloalkyl as used herein means a ring containing from three to eight carbon atoms and includes cyclopropyl, cyclohexyl and the like.

The term heterocycloalkyl as used herein means a 3, 4, 5, 6, 7 or 8-membered ring containing up to two heteroatoms selected from the group consisting of N, S and 0, and includes piperidinyl, piperazinyl, thiopyranyl and the like.

The term alkoxy as used herein means an oxygen radical substituted with a straight- or branched-chain alkyl group containing 1, 2, 3, 4, 5, or 6 carbon atoms and includes methoxy, ethoxy and the like.

The term alkyloxy as used herein means straight and branched-chain alkyl radicals of 1, 2, 3, 4, 5 or 6 carbons substituted with an oxygen atom.

The terms aralkyl and aryloxyalkyl as used herein mean an alkyl radical substituted with an aryl group or aryloxy group, respectively, and including benzyl, phenethyl, 2-phenoxyethyl and the like.

The terms aralkoxy and aryloxyalkoxy as used herein mean an alkoxy radical substituted with an aryl group or arlyoxy group, respectively, and include, benzyloxy, phenoxyethoxy and the like

The terms cycloalkyl-substituted alkyl, cycloalkyl-substituted alkoxy, heterocycloalkyl-substituted alkyl and heterocycloalkyl-substituted alkoxy mean groups such as 2-cyclohexyl-ethyl, 2-cyclohexyl-ethoxy and the like.

The terms alkylene (—CH2—CH2—), alkenylene (—CH=CH—) and alkynylene (—CH=CH—) as used herein means straight- and branched-chain bivalent radicals containingl, 2, 3, 4, 5 and 6-carbon atoms, such as methylene, ethylene, vinylene, propenylene and ethynylene.

The term thioalkyl as used herein means straight- and branched-chain alkyl radicals containing 1, 2, 3, 4, 5 or 6 carbon atoms substituted with SH and includes thiomethyl, thiopropyl and the like.

The term alkanoyl as used herein means a carbonyl substituted with straight- or branched-chain radicals containing 1, 2, 3, 4, 5 or 6 carbon atoms and includes acetyl, propionyl and the like.

The term halo as used herein means halogen and includes fluoro, chloro, bromo, iodo. The term haloalkyl refers to an alkyl group substituted by one or more independently selected halo atoms, such as —CF3.

The term haloalkoxy refers to an alkoxy group substituted by one or more independently selected halo atoms, such as —OCF3.

### DETAILED DESCRIPTION AND PREFERRED EMBODIMENTS

The invention includes 3-aminopiperidine and 4-aminopiperidine compounds defined in Formula 1. Compounds of Formula I include those in which Ar₁, Ar₂ and Ar₃ are, independently, optionally-substituted aryl groups. Embodiments of the invention include those in which the aryl groups Ar₁, Ar₂ and Ar₃ are selected from monocyclic and benzo fused aromatic and heteroaromatic rings such as phenyl, pyridyl, furyl, thienyl, naphthyl, indanyl, quinolinyl, fluorenyl and the like. In a preferred embodiment, Ar₁, Ar₂ and Ar₃ are optionally-substituted phenyl wherein the optional substituents may be selected from alkyl, alkoxy, cycloalkyl, cycloalkyloxy, heterocycloalkyl, heterocycloalkyloxy, alkanoyl, thioalkyl, aralkyl, aralkoxy, aryloxyalkyl, aryloxyalkoxy, cycloalkyl-substituted alkyl, cycloalkyloxy-substituted alkyl, cycloalkyl-substituted alkoxy, cycloalkyloxy-substituted alkoxy, heterocycloalkyl-substituted alkyl, heterocycloalkyloxy-substituted alkyl heterocycloalkyl-substituted alkoxy, heterocycloalkyloxy-substituted alkoxy, thioaryl, aralkylthio, thioaryl-alky, halo, N0₂, Ph, CF₃, OCF₃, CN, OH, methylenedioxy, ethylenedioxy, SO₂NRR″, NRR″, CO₂R (where R and R″ are independently selected from the group consisting of H and alkyl) and aryl.

The aryl subsfituent may be further substituted with a substituent selected from the preceding list;

In a preferred embodiment of the invention Ar₁ is thiophene preferably 3-thiophene. In a more preferred embodiment of the invention Ar₁ is unsubstituted phenyl. In a further preferred embodiment Ar₁ is alkyl-, alkoxy- or halo-substituted phenyl. In a most preferred embodiment of the invention Ar₁ is 2-substituted phenyl wherein the substituent is selected from methoxy, ethoxy and F, such as 2-methoxy phenyl.

Ar₂ is, preferably, an alkoxy-, alkyl-, halo-, or haloalkoxy-substituted phenyl group. More preferably, Ar₂ is an alkoxy or haloalkoxy substituted phenyl group. Most preferably Ar₂ is methoxy-, ethoxy- or trifluoromethoxy-phenyl. The most preferred compounds are those in which the phenyl group is substituted in the 3- or 4-position, such as 3-methoxyphenyl.

Ar₃ is, preferably, an alkoxy-, alkyl- or halo-substituted phenyl group. More preferably, Ar₃ is an alkyl substituted phenyl group. Most preferably Ar₃ is an alkoxy substituted phenyl group. The most preferred compounds are those in which the phenyl group is substituted in the 4-position, such as 4-methoxyphenyl.

Compounds of Formula I include those in which R₁, R₂ and R, are independently selected from the group consisting of H and alkyl. Preferably, R₁, R₂ and R₃ are methyl or H. Most preferably, R₁, R₂ and R₃ are H.

Compounds of Formula I include those in which X is selected from the group consisting of C=O and SO₂. Preferably, X is SO₂.

Compounds of Formula 1 include those in which Y is selected from the group consisting of —[CHR₁]ₘ—, —CH₂CH=CH—, and CH₂C=C—; where m is an integer selected from the group consisting of 1, 2, and 3. Preferably Y is —[CHR₁]—ₘ. More preferably Y is —[CHR₁]—ₘ, where R₁ is H and m is 1.

Embodiments of the invention include those in which n is an integer selected from 0, 1 and 2. Preferably, n is 0 or 1 and, more preferably, n is 0.

Suitable embodiments of the invention include those in which p is an integer selected from the group consisting of 0, 1 and 2. In a preferred embodiment p is 0.

In specific embodiments of the invention, the compounds of Formula I include:

N-Benzoyl-4-[(N-benzyl-N-4-methoxyphenylsulphonyl)amino]-piperidine (1.1);

N-Phenyl-4-[(N-phenyl-N4-methoxyphenylsulphonyl)amino]-piperidine (1.110);

N-Benzyl-4-[(N-2-methoxyphenyl-N-4-methoxyphenylsulphonyl)amino]-piperidine (1.2);

N-Benzyl-4-[(N-benzyl-N-4-methoxyphenylsulphonyl)amino]-piperidine (1.3);

N-Benzyl-4-[(N-2-phenylethyl-N-(4-methoxyphenylsulphonyl)amino]-piperidine (1.4);

N-Benzyl-4-[(N-phenyl)-N-(4-methoxyphenyl)acetyl)amino]-piperidine (1.5);

N-Benzyl-4-[(N-phenyl)-N-(phenoxy)acetyl)amino]-piperidine (1.6);

N-Benzyl-4-[(N-4-methoxybenzyi-N-4-methoxyphenylsulphonyl)amino]-piperidine (1.9);

N-Benzyl-4-[(N-phenyl)-N-((2-benzo[bjthiophenyl)carbonyl)amino]-piperidine (1.7);

N-Benzyl-4-[(N-phenyl-N-4-methoxyphenylsulphonyl)amino]-piperidine (1.8);

N-Benzyl-4-[(N-4-methoxybenzyl-N-phenylsulphonyl)amino]-piperidine (1.10);

N-Benzyl-4-[(N-4-methoxybenzyl-N-4-chlorophenylsulphonyl)amino]-piperidine (1. 11);

N-Benzyl-4-[(N-4-methoxybenzyl-N-4-methylphenylsulphonyl)amino]-piperidine (1.12);

N-Benzyl-4-[(N-4-methoxybenzyl-N-4-phenylphenylsulphonyl)amino]-piperidine (1.13);

N-Benzyl-4-[(N-4-methoxybenzyl-N-2, 5-dimethoxyphenylsulphonyl)amino]-piperidine (1.14);

N-Benzyl-4-[(N-2-phenylethyl-N-phenylsulphonyl)amino]-piperidine (1.15);

N-Benzyl-4-[(N-2-phenylethyl-N-(4-chlorophenyl)sulphonyl)amino]-piperidine (1.16);

N-Benzyl-4-[(N-2-phenylethyl-N-(4-methylphenyl)sulphonyl)amino]-piperidine (1.17);

N-Benzyl-4-[(N-2-phenylethyl-N-(4-phenylphenyl)sulphonyl)amino]-piperidine (1.18);

N-Benzyl-4-[(N-2-phenylethyl-N-(2, 5-dimethoxyphenyl)sulphonyl)amino]-piperidine (1.1 9);

N-Benzyl-4-[(N-phenyl-N-phenylsulphonyl)amino]-piperidine (1.20);

N-Benzyl-4-[(N-phenyl-N-(4-chlorophenyl)sulphonyl)amino]-piperidine (1.21);

N-Benzyl-4-[(N-phenyl-N-(4-methylphenyl)sulphonyl)amino]-piperidine (1.22);

N-Benzyl-4-[(N-phenyl-N-(4-phenylphenyl)sulphonyl)amino]-piperidine (1.23);

N-Benzyl-4-[(N-phenyl-N-(2,5-dimethoxyphenyl)sulphonyl)amino]-piperidine (1.24);

N-((2-furyl)methyl)-4-[(N-benzyl-N-4-methoxypheny/sulphonyl)amino]-piperidine (1.112);

N-Benzyl-4-[(N-((2-furyl)methyl)-N-(4-methoxyphenyl)sulphonyl)amino]-piperldine (1 .25);

N-Benzyl-4-[(N-(4-chlorobenzyl)-N-(4-methoxyphenyl)sulphonyl)amino]-piperidine (1.26);

N-Benzyl-4-[(N-2-chlorobenzyl)-N-(4-methoxyphenyl)sulphonyl)amino]piperidine (1.27);

N-Benzyl-4-[(N-(2-naphthyl)methyl)-N-(4-methoxyphenyl)sulphonyl)amino]-piperidine (1.28);

N-Benzyl-4-[(N-1-naphthylmethyl)-N-(4-methoxyphenyl)sulphonyl)amino]-piperidine (1.29);

N-Benzyl-4-[(N-(4-mothoxyphenyl)-N-(4-methoxyphenyl)sulphonyl)amino]-piperidine (1.30);

N-Benzyl-4-[(N-4-chlorophenyl)-N-(4-methoxyphenyl)sulphonyl)amino]-piperidine (1.31);

N-(2-phenethyl)-4-[(N-phenyl-N-(4-methoxyphenyl)sulphonyl)amino]-piperidine (1.1111 );

N-Benzyl-4-[(N-4-phenylphenyl)-N-(4-methoxyphenyl)sulphonyl)amino]-piperidine (1.32);

N-Benzyl-4-[(N-(2-naphthy)-N-(4-mothoxyphenyl)sulphonyl)amino]-pipelidine (1.33);

N-Benzyl-4-[(N-3-methoxyphenyl)-N-(4-methoxyphenyl)sulphonyl)amino]-piperidine (1.34);

N-Benzyl-4-[(N-3-chlorophenyl)-N-(4-methoxyphenyl)sulphonylamino]-piperdine (1.35);

N-Benzyl-4-[(N-4-methylphenyl)-N-(4-methoxyphenyl)sulphonyl)amino]-piperidine (1.36),

N-Benzyl-4-[(N-4-isopropylphenyl)-N-(4-methoxyphenyl)sulphonyl)amino]-piperidine (1.37);

N-Benzyl-4-[(N-4-trifluoromethoxyphenyl)-N-(4-methoxyphenyl)sulphonyl)amino]-piperidine (1.38);

N-Benzyl-4-[(N-(1-naphthyl))-N-(4-methoxyphenyl)sulphonyl)amino]-piperidine (1.39);

N-Benzyl-4-[(N-3-methylphenyl)-N-(4-methoxyphenyl)sulphonyl)amino]-piperidine (1.40);

N-Benzyl-4-[(N-(5-indanyl))-N-(4-methoxyphenyl)sulphonyl)amino]-piperidine (1.41);

N-Benzyl-4-[(N-3,4-dichlorophenyl)-N-(4-methoxyphenyl)sulphonyl)amino]-piperidine (1.42);

N-Benzyl-4-[(N-3-chloro-4-methoxyphenyl)-N-(4-methoxyphenyl)sulphonyl)amino]-piperidine (1.43);

N-Benzyl-4-[(N-3, 4-methylendioxyphenyl)-N-(4-methoxyphenyl)sulphonyl)amino]-piperidine (1.44);

N-(2-fufylmethyl)-4-[(N-3-methoxyphenyl)-N-(4-methoxyphenyl)sulphonyl)amino]-piperidine (1.45);

N-3-chlorobenzyl-4-[(N-3-methoxyphenyl-N-(4-methoxyphenyl)sulphonyl)amino]-piperidine (1.46);

N-(2-Chlorobenzyl)-4-[(N-3-methoxybenzyl)-N-(4-methoxyphenyl)sulphonyl)amino]-piperidine (1.47);

N-2-methylbenzyl-4-[(N-3-methoxyphenyl)-N-(4-methoxyphenyl)sulphonyl)amino]-piperidine (1.48);

N-2-methoxybenzyl-4-[(N-3-methoxyphenyl)-N-(4-methoxyphenyl)sulphonyl)amino]-piperidine (1.49);

N-3-methoxybenzyl-4-[(N-3-methoxyphenyl)-N-(4-methoxyphenyl)sulphonyl)amino]-piperidine (1.50);

N-4-methoxybenzyl-4-[(N-3-methoxyphenyl)-N-(4-methoxyphenyl)sulphonyl)amino]-piperidine (1.51);

N-3-methylbenzyl-4-[(N-3-methoxyphenyl)-N-(4-methoxyphenyl)sulphonyl)amino]-piperidine (1.52);

N-4-methylbenzyl-4-[(N-3-methoxyphenyl)-N-(4-methoxyphenyl)sulphonyl)amino]-piperidine (1.53);

N-4-chlorobenzyl-4-[(N-3-methoxyphonyl)-N-(4-methoxyphenyl)sulphonyl)amino]-piperidine (1.54);

N-benzyl-4-[(N-3, 5-dimethoxyphenyl)-N-(4-methoxyphenyl)sulphonyl)amino]-piperidine (1.55);

N-benzyl-4-[(N-3-trifluoromethoxyphenyl)-N-(4-methoxyphenyl)sulphonyl)amino]-piperidine (1.56);

N-benzyl-4-[(N-3-isopropylphenyl-N-(4-methoxyphenyl)sulphonyl)amino]-piperidine (1.57);

N-benzyl-4-[(N-3-ethoxyphenyl)-N-(4-methoxyphenyl)sulphonyl)amino]-piperidine (1.58);

N-benzyl-4-[(N-3-isopropoxyphenyl)-N-(4-methoxyphenyl)sulphonyl)amino]-pipeidine (1.59);

N-benzyl-4-[(N-3-phenoxyphenyl)-N-(4-methoxyphenyl)sulphonyl)amino] piperidine (1.60);

N-α-methylbenzyl-4-[(N-3-trifluoromethoxyphenyl)-N-(4-methoxyphenyl) sulphonyl)amino]-piperidine (1.61);

N-2-methylbenzyl-4-[(N-3-trifluoromethoxyphenyl)-N-(4-methoxyphenyl) sulphonyl)amino]-piperidine (1.62);

N-2-methoxylbenzyl-4-[(N-3-trifluoromethoxyphenyl)-N-(4-methoxyphenyl) sulphonyl)-amino]piperidine (1.63);

N-2-ethoxybenzyl-4-[(N-3-trifluoromethoxyphenyl)-N-(4-methoxyphenyl) sulphonyl)amino]-piperidine (1.64);

(R,S)-(trans)-N-Benzyl-3-methyl-4-[(N-phenyl-N-(4-methoxyphenyl)sulphonyl) amino]-piperidine (1.66)

(R,S)-(cis)-N-Benzyl-3-methyl-4-[(N-(3-methoxyphenyl)-N-(4-methoxyphenyl) sulphonyl)-amino]piperidine (1.67)

N-Benzyl-3-[(N-3-methoxyphenyl-N-4-methoxyphenylsulphonyl)amino] piperidine (1.70);

N-Benzyl-3-[(N-3-methoxyphenyl-N-phenylsulphonyl)aminomethyl]piperidine (1.71);

N-Benzyl-3-[(N-3-methoxyphenyl-N-4-methoxyphenylsulphonyl)aminomethyl) piperidine(1.72);

N-2-Cyanobenzyl-4-[(N-3-methoxyphenyl)-N-(4-methoxyphenyl)sulphonyl) amino]piperidine (1.73);

N-2-Nitrobenzyl-4-[(N-3-methoxyphenyl)-N-(4-methoxyphenyl)sulphonyl) amino]piperidine (1.74);

N-2-Fluorobenzyl4-[(N-3-methoxyphenyl)-N-(4-methoxyphenyl)sulphonyl) amino]piperidine (1.75);

N-3-Fluorobenzyl4-[(N-3-methoxyphenyl)-N-(4-methoxyphenyl)sulphonyl) amino]piperidine (1.76);

N-4-Fluorobenzyl-4-[(N-3-methoxyphenyl)-N-(4-methoxyphenyl)sulphonyl) amino]piperidine (1.77);

N-3, 5-Difluorobenzyl-4-[(N-3-methoxyphenyl)-N-(4-methoxyphenyl)sulphonyl) amino]piperidine (1.78);

N-2, 6-Difluorobenzyl4-[(N-3-methoxyphenyl)-N-(4-methoxyphenyl)sulphonyl) amino]piperidine (1.79);

N-Cinnamyl4-[(N-3-methoxyphenyl)-N-(4-methoxyphenyl)sulphonyl) amino]piperidine (1.80);

N-2-Phenylethyl-4-[(N-3-methoxyphenyl)-N-(4-methoxyphenyl)sulphonyl) amino]piperidine (1.81);

N-2-Pyridylmethyl-4-[(N-3-methoxyphenyl)-N-(4-methoxyphenyl)sulphonyl) amino]piperidine (1.82);

N-2-Quinolylmethyl-4-[(N-3-methoxyphenyl)-N-(4-methoxyphenyl)sulphonyl) amino]piperidine (1.83);

N-3-Thienylmethyl-4-[(N-3-methoxyphenyl)-N-(4-methoxyphenyl)sulphonyl) amino]piperidine (1.85);

N-2-Furylmethyl-4-[(N-3-methoxyphenyl)-N-(4-methoxyphenyl)sufphonyl) amino]piperidine (1.85);

N-3-pyridylmethyl-4-[(N-3-methoxyphenyl)-N-(4-methoxyphenyl)sulphonyl) amino]piperidine (1.86);

N-2-ethoxybenzyl-4-[(N-3-methoxyphenyl)-N-(4-methoxyphenyl)sulphonyl) amino]piperidine (1.87);

N-2,3-methylendioxybenzyl-4-[(N-3-methoxyphenyl)-N-(4-methoxyphenyl)sulphonyl)amino]piperidine (1.88);

N-2-Methoxybenzyl-4-[(N-3-methoxyphenyl-N-2, 5-dimethoxy-4-nitrophenylsulphonyl)amino]piperidine (1.89);

N-benzyl-4-[N-(3-(trifluoromethoxy)phenyl)-N-(4-fluorophenylsulphonyl) amino]-piperidine (1.90);

N-benzyl-4-[N-(phenyl)-N-(3-methoxyphenylsulphonyl)amino]-piperidine (1.91);

N-benzyl-4-[N-(phenyl)-N-(4-(trifluoromethoxy)phenylsulphonylvamino]-piperidine (1.92);

N-benzyl-4-[N-(4-methylphenyl)-N-(3-methoxyphenylsulphonyl)amino]-piperidine (1.93);

N-benzyl-4-[N-(4-methylphenyl)-N-(4-(trifluoromethoxy)phenylsulphonyl) amino]-piperidine (1.94);

N-benzyl-4-[N-(4-methylphenyl)-N-(2-naphthylsulphonyl)amino]-piperidine (1.95);

N-benzyl-4-[N-(3-(trifluoromethoxy)phenyl)-N-(3-methoxyphenylsulphonyl) amino]-piperidine (1.96);

N-benzyl-4-[N-(3-trifluoromethoxy)phenyl)-N-(2-cyanophenylsulphonyl) amino]-piperidine (1.97);

N-benzyl-4-[N-(3-trifluoromethoxy)phenyl)-N-(3-cyanophenylsulphonyl) amino]-piperidine (1.98);

N-benzyl-4-[N-(3-trifluoromethoxy)phenyl)-N-(2-naphthylsulphonyl)amino]-piperidine (1.99);

N-(pyridin-4-ylmethyl)-4-[N-(3-trifluoromethoxy)phenyl)-N-(4-methoxyphenylsulphonyl)amino]-piperidine (1.100);

N-(2-chlorobenzyl)-4-[N-(3-trifluoromethoxy)phenyl)-N-(4-methoxyphenylsulphonyl)amino]-piperidine (1.101);

N-benzyl-4-JN-(3-trifluoromethoxy)phenyl)-N-(4-methylphenylsulphonyl) amino]-piperidine (1.102);

N-benzyl-4-[N-(3-trifluoromethoxy)phenyl)-N-(4-tert-butyiphenylsulphonyl) amino]-piperidine (1.1 03);

N-benzyl-4-[(4-t-butylphenyl-N-4-methoxyphenylsulphonyl)aminoapiperidine (1.105);

N-benzyl-4-[(4-trifluoromethyl-N-4-methoxyphenylsulphonyl)amino] piperidine (1.106);

N-benzyl-4-[(3-trifluoromethyl-N-4- methoxyphenylsulphonyl)amino] piperidine (1.107);

N-benzyl-4-[(4-propylphenyl-N-4- methoxyphenylsulphonyl)amino] piperidine (1.108) and N-benzyl-4[(N-3trifluoromethoxyphenyl-N-4-hydroxyphenylsulphonyl) amino]piperdine (1.109).

In another embodiment of the invention, the compound of Formula I is provided in labeled form, such as radiolabeled form (e.g. labeled by incorporation within its structure ³H or ¹⁴C or by conjugation to ¹²⁵I). In a preferred aspect of the invention, such compounds, which bind preferentially to GlyT-1, can be used to identify GlyT-1 receptor ligands by techniques common in the art. This can be achieved by incubating the receptor or tissue in the presence of a ligand -candidate and then incubating the resulting preparation with an equimolar amount of radiolabeled compound of the invention. GlyT-1 receptor ligands are thus revealed as those that significantly occupy the GlyT-1 site and prevent binding of the radiolabeled compound of the present invention. Alternatively, GlyT-1 receptor ligand candidates may be identified by first incubating a radiolabeled form of a compound of the invention then incubating the resulting preparation in the presence of the candidate ligand. A more potent GlyT-1 receptor ligand will, at equimolar concentration, displace the radiolabeled compound of the invention.

Acid addition salts of the compounds of Formula I are most suitably formed from pharmaceutically acceptable acids, and include for example those formed with inorganic acids e.g. hydrochloric, sulphuric or phosphoric acids and organic acids e.g. succinic, maleic, acetic or fumaric acid. Other non-pharmaceutically acceptable salts e.g. oxalates may be used for example in the isolation of compounds of Formula I for laboratory use, or for subsequent conversion to a pharmaceutically acceptable acid addition salt. Also included within the scope of the invention are base addition salts (such as sodium, potassium and ammonium salts), solvates and hydrates of compounds of the invention. The conversion of a given compound salt to a desired compound salt is achieved by applying standard techniques, well known to one skilled in the art.

The compounds of the present invention can be prepared by processes analogous to those established in the art.

N-Benzyl piperidines of Formula I in which X is an SO₂ group and n is 0 are readily prepared by the method shown in Scheme 1, below. Reductive amination, procedure (A), of N-Benzyl-4-piperidone, a, gave intermediate b which, upon treatment with an appropriate arylsulphonyl chloride, procedure (C), gave product c. Compounds in which X is a C=O group can be prepared in an analogous manner. Treatment of amine b with the appropriate acid chloride in the presence of a base such as N,N-diisopropylethylamine procedure (B) will yeild product d.

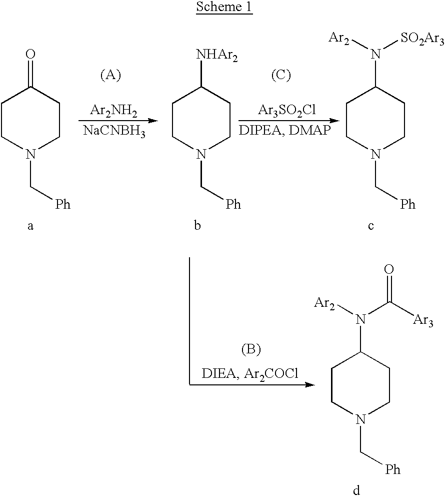

In a similar manner, 3-aminopiperidine compounds of Formula I can be prepared from N-benzyl-3-piperidone, a′, as shown in Scheme 1a below.

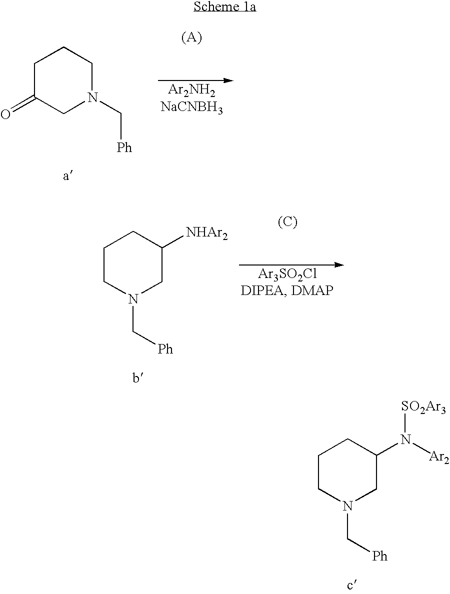

Compound c can also used as an intermediate in the synthesis of other N-aralkyl compounds of the invention, as shown in Scheme 2, below. Debenzylation of compound c by catalytic hydrogenation, procedure (D), gave intermediate d, which, upon treatment with an appropriate aralkyl halide, procedure (E), provided N-aralkyl piperidines e. Compounds of the formula f can be made by reductive amination, procedure (A), of compound d with an arylaldehyde.

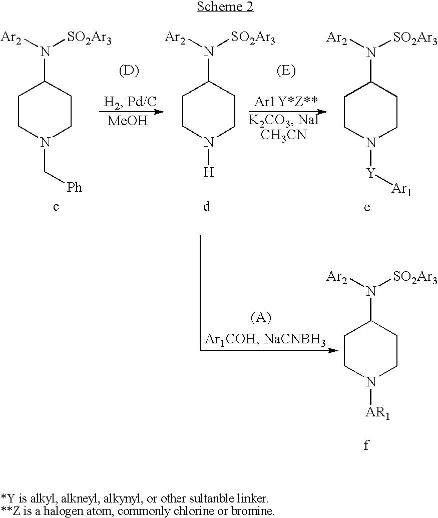

Compounds of Formula I containing the 3-aminomethyl moiety can be made by the method outlined in Scheme 3 below.

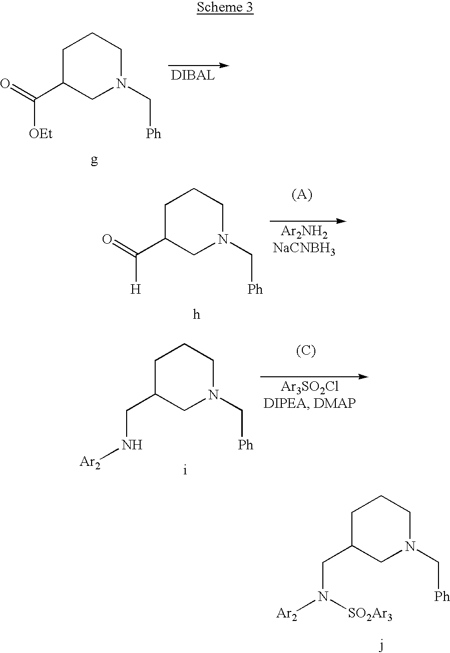

Alternatively, compounds of the invention may also be prepared by solid-phase synthesis, according to the route shown in Scheme 4, below. This parallel-synthesis route has the advantage that it can be used to efficiently prepare a wide number of compounds of the invention. Alkenyl-copolystyrene resin k (REM resin 01-64-0302; novabiochem/Cedarlane Laboratories Limited, Ontario, Canada) upon treatment with piperidone I in the presence of a base (such as Hunig's base) gave intermediate m which, upon reductive amination with Ar₂NH₂ gave intermediate n. Sulphonylation under standard conditions gave the final intermediate o. Quaternization of this intermediate with benzylamine Ar₁CH₂Br followed by treatment with Hunig's base cleaved product e from the resin.

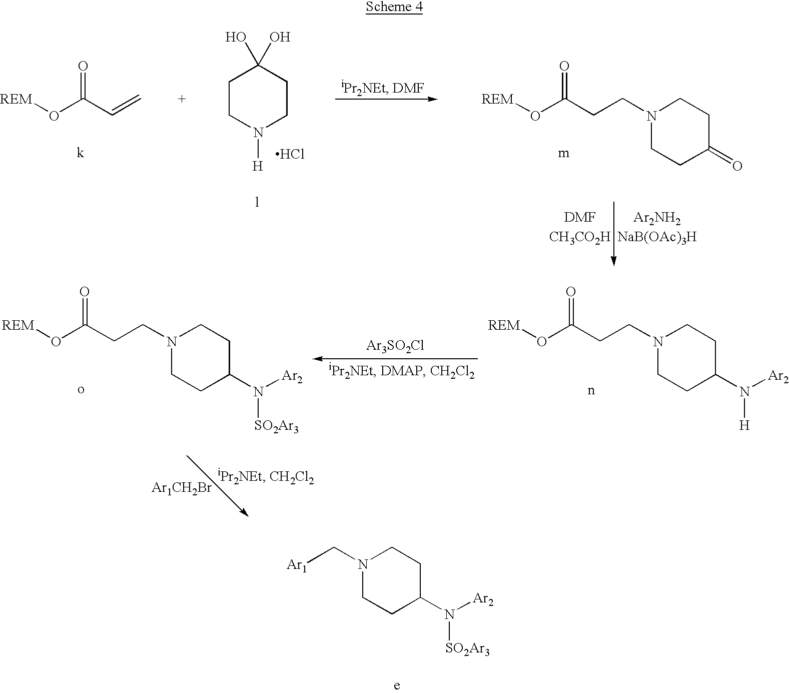

Schemes 1-4 above are provided as general synthetic routes to many of the compounds of the claimed invention. Some compounds of the claimed invention have been made by alternate routes as seen in the examples below.

Compounds which inhibit GlyT-1 mediated glycine transport will increase glycine concentrations at NMDA receptors, which receptors are located in the forebrain, among other locations. This concentration increase elevates the activity of NMDA receptors, thereby alleviating schizophrenia and enhancing cognitive function.

The compounds of the invention are, for instance, administered orally, sublingually, rectally, nasally, vaginally, topically (including the use of a patch or other transdermal delivery device), by pulmonary route by use of an aerosol, or parenterally, including, for example, intramuscularly, subcutaneously, intraperitoneally, intraarterially, intravenously or intrathecally. Administration can be by means of a pump for periodic or continuous delivery. The compounds of the invention are administered alone, or are combined with a pharmaceutically-acceptable carrier or excipient according to standard pharmaceutical practice. For the oral mode of administration, the compounds of the invention are used in the form of tablets, capsules, lozenges, chewing gum, troches, powders, syrups, elixirs, aqueous solutions and suspensions, and the like. In the case of tablets, carriers that are used include lactose, sodium citrate and salts of phosphoric acid. Various disintegrants such as starch, and lubricating agents such as magnesium stearate and talc, are commonly used in tablets. For oral administration in capsule form, useful diluents are lactose and high molecular weight polyethylene glycols. If desired, certain sweetening and/or flavoring agents are added. For parenteral administration, sterile solutions of the compounds of the invention are usually prepared, and the pHs of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. Such compositions can include mucomimetics such as hyaluronic acid, chondroitin sulphate, hydroxypropylmethylcellulose or polyvinyl alcohol, preservatives such as sorbic acid, EDTA or benzylchromium chloride, and the usual quantities of diluents and/or carriers. For pulmonary administration, diluents and/or carriers will be selected to be appropriate to allow the formation of an aerosol.

Suppository forms of the compounds of the invention are useful for vaginal, urethral and rectal administrations. Such suppositories will generally be constructed of a mixture of substances that is solid at room temperature but melts at body temperature. The substances commonly used to create such vehicles include theobroma oil, glycerinated gelatin, hydrogenated vegetable oils, mixtures of polyethylene glycols of various molecular weight and fatty acid esters of polyethylene glycol. See, Remington's Pharmaceutical Sciences, 16th Ed., Mack Publishing, Easton, Pa., 1980, pp. 1530-1533 for further discussion of suppository dosage forms. Analogous gels or creams can be used for vaginal, urethral and rectal administrations.

Numerous administration vehicles will be apparent to those of ordinary skill in the art, including without limitation slow release formulations, liposomal formulations and polymeric matrices.

Examples of pharmaceutically acceptable acid addition salts for use in the present invention include those derived from mineral acids, such as hydrochloric, hydrobromic, phosphoric, metaphosphoric, nitric and sulphuric acids, and organic acids, such as tartaric, acetic, citric, malic, lactic, fumaric, benzoic, glycolic, gluconic, succinic, p-toluenesulphonic and arylsulphonic acids, for example. Examples of pharmaceutically acceptable base addition salts for use in the present invention include those derived from non-toxic metals such as sodium or potassium, ammonium salts and organoamino salts such as triethylamine salts. Numerous appropriate such salts will be known to those of ordinary skill.

The physician or other health care professional can select the appropriate dose and treatment regimen based on the subject's weight, age, and physical condition. Dosages will generally be selected to maintain a serum level of compounds of the invention between about 0.01 μg/cc and about 1000 μg/cc, preferably between about 0.1 μg/cc and about 100 μg/cc. For parenteral administration, an alternative measure of preferred amount is from about 0.001 mg/kg to about 10 mg/kg (alternatively, from about 0.01 mg/kg to about 10 mg/kg), more preferably from about 0.01 mg/kg to about 1 mg/kg (from about 0.1 mg/kg to about 1 mg/kg), will be administered. For oral administrations, an alternative measure of preferred administration amount is from about 0.001 mg/kg to about 10 mg/kg (from about 0.1 mg/kg to about 10 mg/kg), more preferably from about 0.01 mg/kg to about 1 mg/kg (from about 0.1 mg/kg to about 1 mg/kg). For administrations in suppository form, an alternative measure of preferred administration amount is from about 0.1 mg/kg to about 10 mg/kg, more preferably from about 0.1 mg/kg to about 1 mg/kg.

For use in assaying for activity in inhibiting glycine transport, eukaryokic cells, preferably QT-6 cells derived from quail fibroblasts, have been transfected to express one of the four known variants of human GlyT-1, namely GlyT-1 a, GlyT-1b, GlyT-1c, or Gly T-1d or human GlyT-2. The sequences of GlyT-1a, GlyT-1b and GlyT-1c are described in Kim et al., Molec. Pharm. 45: 608-617, 1994, excepting that the sequence encoding the extreme N-terminal of GlyT-1a was merely inferred from the corresponding rat-erived sequence. This N-terminal protein-encoding sequence has now been confirmed to correspond to that inferred by Kim et al. The sequence of GlyT-1d is described in U.S. Pat. No. 6,008,015. The sequence of the human GlyT-2 is described in U.S. Pat. No. 5,919,653. Suitable expression vectors include pRc/CMV (Invitrogen), Zap Express Vector (Stratagene Cloning Systems, LaJolla, CA; hereinafter “Stratagene”), pBk/CMV or pBk-RSV vectors (Stratagene), Bluescript II SK±Phagemid Vectors (Stratagene), LacSwitch (Stratagene), pMAM and pMAM neo (Clontech), among others. A suitable expression vector is capable of fostering expression of the included GlyT DNA in a suitable host cell, preferably a non-mammalian host cell, which can be eukaryotic, fungal, or prokaryotic. Such preferred host cells include amphibian, avian, fungal, insect, and reptilian cells.

### EXPERIMENTAL EXAMPLES

(A). General Procedure for Reductive Amination with Various Amines. Ketones, and Aldehydes

To an ice-cooled solution of the amine (1.18 mmol) in methanol (4 mL) was added sodium cyanoborohydride (0.6 eq.), the ketone or aldehyde (0.9 eq.), and acetic acid (1.4 eq.). After the addition, the reaction mixture was allowed to warm to room temperature and stirred 16 hours. The mixture was then cooled in an ice bath and quenched with a 40% potassium carbonate solution. After extraction with dichloromethane, the combined organic layers were washed with brine, dried over anhydrous sodium sulphate and concentrated. The residue was then purified by flash column chromatography over silica gel eluted with 5% 2M ammonia in methanol/dichloromethane to afford the desired amine.

(B). General Procedure for Acetylation with an Amine and Various Acid Chlorides

To a solution of the amine (0.20 mmol) in dichloroethane (1 mL) at room temperature was added N,N-diisopropylethylamine (1.3-1.5 eq.) and the acid chloride (1.2-1.5 eq.). The reaction mixture was stirred overnight and then washed with dilute HCl, saturated sodium bicarbonate and brine, dried over anhydrous sodium sulphate and concentrated. The residue was purified by flash column chromatography over silica gel eluted with either 4% 2M ammonia in methanol/dichloromethane or 8-10% acetone/benzene to afford the acetylated product.

(C). General Procedure for Sulphonylation with Various Amines and Benzenesulphonyl Chlorides

To a solution of the amine (0.15 mmol) in 1,2-dichloroethane (1 mL) at room temperature was added N,N-diisopropylethylamine (2.0 eq.), the benzenesulphonyl chloride (2.0 eq.), and 4-dimethylaminopyridine (0.2 eq.). The reaction mixture was then stirred at 70° C. for 5 hours. After cooling the mixture was washed with 1 N HCl, water, saturated sodium bicarbonate and brine, dried over anhydrous sodium sulphate and concentrated. The residue was purified by flash column chromatography over silica gel eluted with 30-45% ethyl acetate/hexane to afford the sulphonylated product.

(D). General Procedure for Debenzylation

To a suspension of 10 wt. % Pd/C (0.9 mg of catalyst/mg of reactant) in methanol (5 mL) was added a solution of the benzyl compound (0.83 mmol) in methanol (20 mL). After the addition, a balloon filled with H₂(g) was attached to the flask. The flask was then evacuated and filled with H₂(g). After 16 hours of stirring the reaction mixture was filtered through celite and concentrated. The residue was purified by flash column chromatography over silica gel eluted with 5% -10% 2M ammonia in methanol/dichloromethane to afford the de-benzylated product.

Debenzylation can also be carried out using 20% Pd(OH)₂/C catalyst (0.5mg/mg of reactant), the reaction being carried out in ethyl acetate.

(E). General Procedure for Alkylations with Various Benzvl bromides

To a solution of the amine (0.11 mmol) in acetonitrile (1 mL) was added potassium carbonate (1.5 eq.), the benzyl bromide (1.5 eq.), and sodium iodide (0.15 eq.). After stirring for 16 hours the reaction mixture was washed with water and brine, dried over anhydrous sodium sulphate and concentrated. The residue was purified by flash column chromatography eluted with 35% ethyl acetate/hexane to afford the desired product.

If necessary the alkylation can be carried out at elevated temperature, for example at 70° C.

1.1 N-Benzoyl-4-[(N-benzyl-N-4-methoxyphenylsulphonyl)amino]piperidine

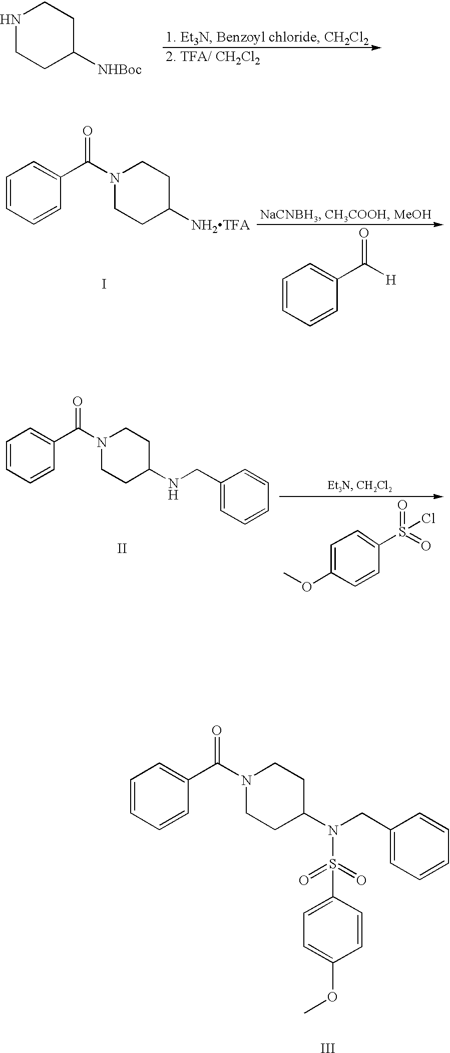

To a solution of 4-N-Boc-amino-piperidine (2.01 mmol) in dry dichloromethane (10 mL) was added triethylamine (2 eq.) and benzoyl chloride (1.1 eq.). After stirring for 30 minutes at room temperature the reaction mixture was concentrated and the residue was placed under high vacuum overnight. The sample was then treated with 50 % TFA/dichloromethane (30 mL) for 30 minutes. The reaction mixture was then concentrated and placed under high vacuum overnight. This crude material was used directly in the next step.

To a solution of the above product (0.18 mmol) in methanol (5 mL) was added sodium cyanoborohydride (1.2 eq.) and then benzaldehyde (1.05 eq.) and the pH was adjusted to 6 by dropwise addition of acetic acid. After stirring for 1 hour the reaction mixture was then concentrated and the crude material used directly in the next.

To a solution of the above product (0.014 mmol) in dichloromethane (1 mL) at room temperature was added triethylamine (5.0 mL) and 4-methxoybenzenesulphonyl chloride (7.5 eq.). After stirring for 16 hours the reaction mixture was concentrated and the residue was purified by flash column chromatography over silica gel eluted first with 33% hexane/ethyl acetate and then with 5% 2M ammonia in methanol/dichloromethane to afford the titled compound as a white solid.

1.2 N-Benzyl-4-[(N-2-methoxyphenyl-N-4-methoxyphenylsulphonyl)amino] piperidine

The above general procedures for reductive amination (A) using o-anisidine and 1-benzyl4-piperidone and then sulphonylation (C) using 4-methoxybenzenesulphonyl chloride were followed. The titled compound was isolated as a white solid (5%).

1.3 N-Benzyl-4-[(N-benzyl-N-4-methoxyphenylsulphonyl)amino]piperidine

The above general procedures for reductive amination (A) using 4-amino-1-benzylpiperidine and benzaldehyde and then sulphonylation (C) using 4-methoxybenzenesulphonyl chloride were followed. The titled compound was isolated as a yellow oil (47 %).

1.4 N-Benzyl-4-[(N-2-phenylethyl-N-(4-methoxyphenylsulphonyl)amino]piperidine

The above general procedures for reductive amination (A) using 4-amino-1-benzylpiperidine and phenylacetaldehyde and then sulphonylation (C) using 4-methoxybenzenesulphonyl chloride were followed. The titled compound was isolated as a white solid (51%).

1.5 N-Benzyl-4-[(N-phenyl)-N-(4-methoxyphenyl)acetyl)amino]piperidine

The above general procedures for reductive amination (A) using aniline and 1-benzyl-4-piperidone and then acetylation (B) using 4-methoxyphenylacetyl chloride were followed. The titled compound was isolated as a yellow oil (14%).

1.6 N-Benzyl-4-[(N-phenyl)-N-(phenoxy)acetyl)aminojpiperidine

The above general procedures for reductive amination (A) using aniline and 1-benzyl-4-piperidone and then acetylation (B) using phenoxyacetyl chloride were followed. The titled compound was isolated as a white solid (24%).

1.7 N-Benzyl-4-[(N-phenyl)-N-((2-benzo[bjthiophenyl)carbonyl)amino]piperidine

The above general procedures for reductive amination (A) using aniline and 1-benzyl-4-piperidone and then acetylation (B) using benzo[b]thiophene-2-carbonyl chloride were followed. The titled compound was isolated as a white solid (23%).

1.8 N-Benzyl-4-[(N-phenyl-N-4-methoxyphenylsulphonyl)amino]piperidine

The above general procedures for reductive amination (A) using aniline and 1-benzyl4-piperidone and then sulphonylation (C) using 4-methoxybenzenesulphonyl chloride were followed. The titled compound was isolated as a white solid (23 %).

1.9 N-Benzyl-4-[(N-4-methoxybenzyl-N-4-methoxyphenylsulphonyl)amino] piperidine

The above general procedures for reductive amination (A) using 4-amino-1-benzylpiperidine and p-anisaldehyde and then sulphonylation (C) using 4-methoxybenzenesulphonyl chloride were followed. The titled compound was isolated as a white solid (50%).

1.10 N-Benzyl-4-[(N-4-methoxybenzyl-N-phenylsulphonyl)amino]piperidine

The above general procedures for reductive amination (A) using 4-amino-1-benzylpiperidine and p-anisaldehyde and then sulphonylation (C) using benzenesulphonyl chloride were followed. The titled compound was isolated as a yellow solid (54%).

1.11 N-Benzyl-4-[(N-4-methoxybenzyl-N-4-chlorophenylsulphonyl)amino] piperidine

The above general procedures for reductive amination (A) using 4-amino-1-benzylpiperidine and p-anisaldehyde and then sulphonylation (C) using 4-chlorobenzenesulphonyl chloride were followed. The titled compound was isolated as a white solid (52%).

1.12 N-Benzyl-4-[(N-4-methoxybenzyl-N-4-methylphenylsulphonyl)amino] piperidine

The above general procedures for reductive amination (A) using 4-amino-1-benzylpiperidine and p-anisaldehyde and then sulphonylation (C) using p-toluenesulphonyl chloride were followed. The titled compound was isolated as a yellow solid (52%).

1.13 N-Benzyl-4-[(N-4-methoxybenzyl-N-4phenylphenylsulphonyl)amino] piperidine

The above general procedures for reductive amination (A) using 4-amino-1-benzylpiperidine and p-anisaldehyde and then sulphonylafion (C) using 4-biphenylsulphonyl chloride were followed. The titled compound was isolated as a white solid (48 %).

1.14 N-Benzyl-4-[(N-4-methoxybenzyl-N-2,5-dimethoxyphenylsulphonyl)amino] piperidine

The above general procedures for reductive amination (A) using 4-amino-1-benzylpiperidine and p-anisaldehyde and then sulphonylation (C) using 2,5-dimethoxybenzenesulphonyl chloride were followed. The titled compound was isolated as a white solid (52%).

1.15 N-Benzyl-4-[(N-2-phenylethyl-N-phenylsulphonyl)amino]piperidine

The above general procedures for reductive amination (A) using 4-amino-1-benzylpiperidine and phenylacetaldehyde and then sulphonylation (C) using benzenesulphonyl chloride were followed. The titled compound was isolated as a yellow solid (36%)

1.16 N-Benzyl-4-[(N-2-phenylethyl-N-(4-chlorophenyl)sulphonyl)amino]piperidine

The above general procedures for reductive amination (A) using 4-amino-1-benzylpiperidine and phenylacetaldehyde and then sulphonylation (C) using 4-chlorobenzenesulphonyl chloride were followed. The titled compound was isolated as a yellow solid (47%).

1.17 N-Benzyl-4-[(N-2-phenylethyl-N-(4-methylphenyl)sulphonyl)amino]piperidine

The above general procedures for reductive amination (A) using 4-amino-1-benzylpiperidine and phenylacetaldehyde and then sulphonylation (C) using p-toluenesulphonyl chloride were followed. The titled compound was isolated as a yellow solid (47%).

1.18 N-Benzyl-4-[(N-2-phenylethyl-N-(4-phenylphenyl)sulphonyl)amino]piperidine

The above general procedures for reductive amination (A) using 4-amino-1-benzylpiperidine and phenylacetaldehyde and then sulphonylation (C) using 4-biphenylsulphonyl chloride were followed. The titled compound was isolated as a white solid (25%).

1.19 N-Benzyl-4-[(N-2-phenylethyl-N-(2,5-dimethoxyphenyl)sulphonyl)amino] piperidine

The above general procedures for reductive amination (A) using 4-amino-1-piperidine and phenylacetaldehyde and then sulphonylation (C) using 2,5-dimethoxybenzenesulphonyl chloride were followed. The titled compound was isolated as a white solid (53%).

1.20 N-Benzyl-4-[(N-phenyl-N-phenylsulphonyl)amino]piperidine

The above general procedures for reductive amination (A) using aniline and 1-benzyl-4-piperidone and then sulphonylation (C) using benzenesulphonyl chloride were followed. The titled compound was isolated as a yellow solid (32%).

1.21 N-Benzyl-4-[(N-phenyl-N-(4-chlorophenyl)sulphonyl)amino]piperidine

The above general procedures for reductive amination (A) using aniline and 1-benzyl-4-piperidone and then sulphonylation (C) using 4-chlorobenzenesulphonyl chloride were followed. The titled compound was isolated as a white solid (30%).

1.22 N-Benzyl-4-[(N-phenyl-N-(4-methylphenyl)sulphonyl)amino]piperidine

The above general procedures for reductive amination (A) using aniline and 1-benzyl-4-piperidone and then sulphonylation (C) using p-toluenesulphonyl chloride were followed. The titled compound was isolated as a white solid (33%).

1.23 N-Benzyl-4-[(N-phenyl-N-(4-phenylphenyl)sulphonyl)amino]piperidine The above general procedures for reductive amination (A) using aniline and 1-benzyl-4-piperidone and then sulphonylation (C) using 4-biphenylsulphonyl chloride were lo followed. The titled compound was isolated as a white solid (18%).

1.24 N-Benzyi-4-[(N-phenyl-N-(2,5-dimethoxyphenyl)sulphonyl)amino]piperidine

The above general procedures for reductive amination (A) using aniline and 1-benzyl-4-piperidone and then sulphonylation (C) using 2,5-dimethoxybenzenesulphonyl chloride were followed. The titled compound was isolated as a white solid (25%).

1.25 N-Benzyl-4-[(N-((2-furyl)methyl)-N-(4-methoxyphenyl)sulphonyl)amino]piperidine

The above general procedures for reductive amination (A) using 4-amino-1-benzylpiperidine and 2-thiophenecarboxaldehyde and then sulphonylation (C) using 4-methoxybenzenesulphonyl chloride were followed. The titled compound was isolated as a yellow oil (94%).

1.26 N-Benzyl-4-[(N-(4-chlorobenzyl)-N-(4-methoxyphenyl)sulphonyl)amino] piperidine

The above general procedures for reductive amination (A) using 4-amino-1-benzylpiperidine and 4-chlorobenzaldehyde and then sulphonylation (C) using 4-methoxybenzenesulphonyl chloride were followed. The titled compound was isolated as a colourless oil (65%).

1.27 N-Benzyl-4-[(N-2-chlorobenzyl)-N-(4-methoxyphenyl)sulphonyl)amino] piperidine

The above general procedures for reductive amination (A) using 4-amino-1-benzylpiperidine and 2-chlorobenzaldehyde and then sulphonylation (C) using 4-methoxybenzenesulphonyl chloride were followed. The titled compound was isolated as a yellow oil (59%).

1.28 N-Benzyl-4-[(N-(2-naphthyl)methyl)-N-(4-methoxyphenyl)sulphonyl)amino] piperidine

The above general procedures for reductive amination (A) using 4-amino-1-benzylpiperidine and 2-naphthaldehyde and then sulphonylation (C) using 4-methoxybenzenesulphonyl chloride were followed. The titled compound was isolated as a white solid (79%).

1.29 N-Benzyl-4-[(N-1-naphthylmethyl)-N-(4-methoxyphenyl)sulphonyl)amino] piperidine

The above general procedures for reductive amination (A) using 4-amino-1-benzylpiperidine and 1-naphthaldehyde and then sulphonylation (C) using 4-methoxybenzenesulphonyl chloride were followed. The titled compound was isolated as a white solid (74%).

1.30 N-Benzyl-4-[(N-(4-methoxyphenyl)-N-(4-methoxyphenyl)sulphonyl)amino] piperidine

The above general procedures for reductive amination (A) using p-anisidine and 1-benzyl-4-piperidone and then sulphonylation (C) using 4-methoxybenzenesulphonyl chloride were followed. The titled compound was isolated as a white solid (46%).

1.31 N-Benzyl-4-[(N-4-chlorophenyl)-N-(4-methoxyphenyl)sulphonyl)amino] piperidine

The above general procedures for reductive amination (A) using 4-chloroaniline and 1-benzyl-4-piperidone and then sulphonylation (C) using 4-methoxybenzenesulphonyl chloride were followed. The titled compound was isolated as a white solid (9%).

1.32 N-Benzyl-4-[(N-4-phenylphenyl)-N-(4-methoxyphenyl)sulphonyl)amino] piperidine

The above general procedures for reductive amination (A) using 4-aminobiphenyl and 1-benzyl4-piperidone and then sulphonylation (C) using 4-methoxybenzenesulphonyl chloride were followed. The titled compound was isolated as a white solid (24%).

1.33 N-Benzyl-4-[(N-(2-naphthyl)-N-(4-methoxyphenyl)sulphonyl)amino]piperidine

The above general procedures for reductive amination (A) using 2-aminonaphthalene and 1 -benzyl-4-piperidone and then sulphonylation (C) using 4-methoxybenzenesulphonyl chloride were followed. The titled compound was isolated as a white solid (26%).

1.34 N-Benzyl-4-[(N-3-methoxyphenyl)-N-(4-methoxyphenyl)sulphonyl)amino] piperidine

The above general procedures for reductive amination (A) using m-anisidine and 1-benzyl-4-piperidone and then sulphonylation (C) using 4-methoxybenzenesulphonyl chloride were followed. The titled compound was isolated as a yellow solid (35%).

1.35 N-Benzyl-4-[(N-3-chlorophenyl)-N-(4-methoxyphenyl)sulphonyl)amino] piperidine

The above general procedures for reductive amination (A) using 3-chloroaniline and 1-benzyl-4-piperidone and then sulphonylation (C) using 4-methoxybenzenesulphonyl chloride were followed. The titled compound was isolated as a white solid (12%).

1.36 N-Benzyl-4-[(N-4-methylphenyl)-N-(4-methoxyphenyl)sulphonyl)amino] piperidine

The above general procedures for reductive amination (A) using p-toluidine and 1-benzyl-4-piperidone and then sulphonylation (C) using 4-methoxybenzenesulphonyl chloride were followed. The titled compound was isolated as a white solid (43%).

1.37 N-Benzyl-4-[(N-4-isopropylphenyl)-N-(4-methoxyphenyl)sulphonyl)amino]piperidine

The above general procedures for reductive amination (A) using 4-isopropylaniline and 1-benzyl-4-piperidone and then sulphonylation (C) using 4-methoxybenzenesulphonyl chloride were followed. The titled compound was isolated as a white solid (58%).

1.38 N-Benzyl-4-[(N-4-trifluoromethoxyphenyl)-N-(4 methoxyphenyl)sulphonyl]) amino]piperidine

The above general procedures for reductive amination (A) using 4-(trifluoromethoxy)-aniline and 1-benzyl-4-piperidone and then sulphonylation (C) using 4-methoxybenzenesulphonyl chloride were followed. The titled compound was isolated as a colourless oil (16%).

1.39 N-Benzyl-4-[(N-(1-naphthyl))-N-(4-mothoxyphonyl)sulphonyl)amino]piperidine

The above general procedures for reductive amination (A) using 1-aminonaphthalene and 1-benzyl-4-piperidone and then sulphonylation (C) using 4-methoxybenzenesulphonyl chloride were followed. The titled compound was isolated as a light brown solid (15%).

1.40 N-Benzyl-4-[(N-3-methylphenyl)-N-(4-methoxyphenyl)sulphonyl)amino] piperidine

The above general procedures for reductive amination (A) using m-toluidine and 1-benzyl-4-piperidone and then sulphonylation (C) using 4-methoxybenzenesulphonyl chloride were followed. The titled compound was isolated as a yellow solid (40%).

1.41 N-Benzyl-4-[(N-(5-indanyl))-N-(4-methoxyphenylsulphonyl)amino]piperidine

The above general procedures for reductive amination (A) using 5-aminoindan and 1-benzyl-4-piperidone and then sulphonylation (C) using 4-methoxybenzenesulphonyl chloride were followed. The titled compound was isolated as a yellow solid (50%).

1.42 N-Benzyl-4-[(N-3,4-dichlorophenyl)-N-(4-methoxyphenyl)sulphonyl)amino] piperidine

The above general procedures for reductive amination (A) using 3,4-dichloroaniline and 1-benzyl-4-piperidone and then sulphonylation (C) using 4-methoxybenzenesulphonyl chloride were followed. The titled compound was isolated as a yellow solid (16%).

1.43 N-Benzyl-4-[(N-3-chloro-4-methoxyphenyl)-N-(4-methoxyphenyl)sulphonyl) amino]piperidine

The above general procedures for reductive amination (A) using 3-chloro-4-methoxyaniline and 1-benzyl4-piperidone and then sulphonylation (C) using 4-methoxybenzenesulphonyl chloride were followed. The titled compound was isolated as a white solid (48%).

1.44 N-Benzyl4-[(N-3,4-methylendloxyphenyl)-N-(4-methoxyphenyl)sulphonyl) amino]piperidine

The above general procedures for reductive amination (A) using 3,4-(methylenedioxy)aniline and 1-benzyl4-piperidone and then sulphonylation (C) using 4-methoxybenzenesulphonyl chloride were followed. The titled compound was isolated as a white solid (61%).

1.45 N-(2-furylmethyl)-4-[(N-3-mothoxyphenyl)-N-(4-methoxyphenyl)sulphonyl) amino]piperidine

The titled compound was prepared by following the above procedures for de-benzylation (D) of compound 1.34 and then reductive amination (A) using 2-thiophenecarboxaldehyde and isolated as a white solid (29%).

1.46 N-3-chlorobenzyi-4-[(N-3-methoxyphenyl)-N-(4-methoxyphenyl)sulphonyl) amino]piperidine

The titled compound was prepared by following the above procedures for de-benzylation (D) of compound 1.34 and then alkylation (E) using 3-chlorobenzyl bromide and isolated as a white solid (49%).

1.47 N-(2-Chlorobenzyl)-4-[(N-3-methoxyphenyl)-N-(4-methoxyphenyl)sulphonyl) amino]piperidine

The titled compound was prepared by following the above procedures for de-benzylation (D) of compound 1.34 and then alkylation (E) using 2-chlorobenzyl bromide and isolated as a white solid (45%).

1.48 N-2-methylbenzyl-4-[(N-3-methoxyphenyl)-N-(4methoxyphenyl)sulphonyl) amino]piperidine

The titled compound was prepared by following the above procedures for de-benzylation (D) of compound 1.34 and then alkylation (E) using 2-methylbenzyl bromide and isolated as a yellow solid (35%).

1.49 N-2-methoxybenzyi-4-[(N-3-methoxyphenyl)-N-(4-methoxyphenyl)sulphonyl) amino]piperidine

The titled compound was prepared by following the above procedures for de-benzylation (D) of compound 1.34 and then reductive amination (A) using 2-methoxybenzaldehyde and isolated as a yellow solid (45%).

1.50 N-3-methoxybenzyl-4-[(N-3-methoxyphenyl)-N-(4-methoxyphenyl)sulphonyl) amino]piperidine

The titled compound was prepared by following the above procedures for de-benzylation (D) of compound 1.34 and then reductive amination (A) using 3-methoxybenzaldehyde and isolated as a yellow solid (40%).

1.51 N-4-methoxybenzyl-4-[(N-3-methoxyphenyl)-N-(4-methoxyphenyl)sulphonyl) amino]piperidine

The titled compound was prepared by following the above procedures for de-benzylation (D) of compound 1.34 and then reductive amination (A) using 4-methoxybenzaldehyde and isolated as a yellow solid (32%).

1.52 N-3-methylbenzyl-4-[(N-3-methoxyphenyl)-N-(4-methoxyphenyl)sulphonyl) amino]piperidine

The titled compound was prepared by following the above procedures for de-benzylation (D) of compound 1.34 and then reductive amination (A) using 3-methylbenzaldehyde and isolated as a white solid (44%).

1.53 N4-methylbenzyl-4-[(N-3-methoxyphonyl)-N-(4-methoxyphenyl)sulphonyl) amino]piperidine

The titled compound was prepared by following the above procedures for de-benzylation (D) of compound 1.34 and then reductive amination (A) using 4-methylbenzaldehyde and isolated as a white solid (48%).

1.54 N-4-chlorobenzyl-4-[(N-3-methoxyphenyl)-N-(4-methoxyphenyl)sulphonyl) amino]piperidine

The titled compound was prepared by following the above procedures for de-benzylation (D) of compound 1.34 and then alkylation (E) using 4-chlorobenzyl bromide and isolated as a white solid (26%).

1.55 N-benzyl-4-[(N-3,5-dimethoxyphenyl)-N-(4-methoxyphenyl)sulphonyl)amino] piperidine

The above general procedures for reductive amination (A) using 3,5-dimethoxyaniline and 1-benzyl-4-piperidone and then sulphonylation (C) using 4-methoxybenzenesulphonyl chloride were followed. The titled compound was isolated as a white solid (45%).

1.56 N-benzyl-4-[(N-3-trifluoromethoxyphenyl)-N-(4-methoxyphenyl)sulphonyl) amino]piperidine

The above general procedures for reductive amination (A) using 3-(trifluoromethoxy)-aniline and 1-benzyl-4-piperidone and then sulphonylation (C) using 4-methoxybenzenesulphonyl chloride were followed. The titled compound was isolated as a white solid (36%).

1.57 N-benzyl-4-[(N-3-isopropylphenyl)-N-(4-methoxyphenyl)sulphonyl)amino] piperidine

The above general procedures for reductive amination (A) using 3-isopropylaniline and 1-benzyl-4-piperidone and then sulphonylation (C) using 4-methoxybenzenesulphonyl chloride were followed. The titled compound was isolated as a colourless oil (52%).

1.58 N-benzyl4-[(N-3-ethoxyphenyl)-N-(4-methoxyphenyl)sulphonyl)amino piperidine

The above general procedures for reductive amination (A) using 3-ethoxyaniline and 1-benzyl-4-piperidone and then sulphonylation (C) using 4-methoxybenzenesulphonyl chloride were followed. The titled compound was isolated as a white solid (45%).

1.59 N-benzyl-4-[(N-3-isopropoxyphenyl)-N-(4-methoxyphenyl)sulphonyl)amino] piperidine

The above general procedures for reductive amination (A) using 3-isopropoxyaniline and 1-benzyl-4-piperidone and then sulphonylation (C) using 4-methoxybenzenesulphonyl chloride were followed. The titled compound was isolated as a yellow oil (47%).

1.60 N-benzyl-4-[(N-3-phenoxyphenyl)-N-(4-methoxyphenyl)sulphonyl)amino] piperidine

The above general procedures for reductive amination (A) using 3-phenoxyaniline and 1-benzyl4-piperidone and then sulphonylation (C) using 4-methoxybenzenesulphonyl chloride were followed. The titled compound was isolated as a white solid (37%)

1.61 N-α-methylbenzyl-4-[(N-3-trifluoromethoxyphenyl)-N-(4-methoxyphenyl) sulphonyl)amino]-piperidine

The titled compound was prepared by following the above procedures for de-benzylation (D) of N-Benzyl-4-[(N-3-trifluoromethoxyphenyl)-N-(4-methoxyphenyl)-sulphonyl)amino]piperidine and then alkylation (E) using (1-chloroethyl) benzene and isolated as a yellow oil (41%).

1.62 N-2-methylbenzyl-4-[(N-3-trifluoromethoxyphenyl)-N-(4-methoxyphenyl) sulphonyl)amino]-piperidine

The titled compound was prepared by following the above procedures for de-benzylation (D) of N-Benzyl-4-[(N-3-trifluoromethoxyphenyl)-N-(4-methoxyphenyl)-sulphonyl)amino]piperidine and then reductive amination (A) using o-tolualdehyde and isolated as a colorless oil (46%).

1.63 N-2-methoxylbenzyl-4-f(N-3-trifluoromethoxyphenyl)-N-(4-methoxyphenyl) sulphonyl)-amino]piperidine

The titled compound was prepared by following the above procedures for de-benzylation (D) of N-Benzyl4-[(N-3-trifluoromethoxyphenyl)-N-(4-methoxyphenyl)-sulphonyl)amino]piperidine and then reductive amination (A) using o-anisaldehyde and isolated as a white solid (47%).

1.64 N-2-ethoxybenzyl4-[(N-3-trifluoromethoxyphenyl)-N-(4-methoxyphenyl) sulphonyl)amino]-piperidine

The titled compound was prepared by following the above procedures for de-benzylation (D) of N-Benzyl-4-[(N-3-trifluoromethoxyphenyl)-N-(4-methoxyphenyl)-sulphonyl)amino]piperidine and then reductive amination (A) using 2-ethoxybenzaldehyde and isolated as a colorless oil (48%).

1.65 (RS)-(cis)-N-Benzyl-3-methyl-4-[(N-phenyl-N-(4-methoxyphenyl)sulphonyl) amino]piperidine

The above general procedure for reductive amination (A) using aniline and (R,S)-1-benzyl-3-methyl-piperidin4-one and then sulphonylation (C) using 4-methoxybenzene sulphonyl chloride with the less polar cis-aniline were followed. The title compound was isolated as a yellow solid (12%).

1.66 (RS)-(trans)-N-Benzyl-3-methyl-4-[(N-phenyl-N-(4-methoxyphenyl)sulphonyl) amino]-piperidine

The above general procedure for reductive amination (A) using aniline and (R,S)-1-benzyl-3-methyl-piperidin-4-one and then sulphonylation (C) using 4-methoxybenzene sulphonyl chloride with the more polar trans-aniline were followed. The title compound was isolated as a yellow solid (4%).

1.67 (RS)-(cis)-N-Benzyi-3-methyl-4-[(N-(3-methoxyphonyl)-N-(4-methoxyphenyl) sulphonyl)-amino]piperidine

The above general procedure for reductive amination (A) using m-anisidine and (R,S)-1-benzyl-3-methyl-piperidin-4-one and then sulphonylation (C) using 4-methoxybenzene sulphonyl chloride with the less polar cis-aniline were followed. The title compound was isolated as a yellow solid (4%).

1.70 N-Benzyi-3-[(N-3-methoxyphenyl-N-4-methoxyphenylsulphonyl)amino]piperidine

The above general procedures for reductive amination (A) using m-anisidine and 1-benzyl-3-piperidone hydrochloride and then sulphonylation (C) using 4-methoxybenzenesulphonyl chloride were followed. The titled compound was isolated as a beige foam (52%).

1.71 N-Benzyl-3-[(N-3-methoxyphenyl-N-phenylsulphonyl)aminomethyl] piperidine

DIBAL (7.0 mL, 1.5 M in toluene, 10.5 mmol) was added (dropwise along the sides of the flask) to a solution of ethyl 1-benzylpiperidine-3-carboxylate in toluene (from Emka-Chemie, 1.01 g, 4.08 mmol) at −78° C. and the resulting solution was stirred at −78 ° C. for 3.5 hours. The reaction mixture was quenched by slow addition of ethyl acetate (10 mL) and methanol (5 mL) at −78 ° C. After 15 min, a solution of potassium sodium tartrate (1 M aqueous) was added and the resulting precipitate was stirred for ˜1 hour, further diluted with ethyl acetate and filtered to remove the precipitate. The solvent was removed in vacuo, using methanol to aid azeotropic removal of the toluene at the end, providing 1 -benzylpiperidine-3-carboxaldehyde of sufficient purity for use below.

The above general procedures for reductive amination (A) using m-anisidine and 1-benzylpiperidine-3-carboxaldehyde and then sulphonylation (C) using benzenesulphonyl chloride were followed. The titled compound was isolated as a viscous yellow oil (21%).

1.72 N-Benzyl-3-[(N-3-methoxyphenyl-N-4-methoxyphenylsulphonyl)aminomethyl]piperidine

The above general procedures for reductive amination (A) using m-anisidine and 1-benzylpiperidine-3-carboxaldehyde (prepared above) and then sulphonylation (C) using 4-methoxybenzenesulphonyl chloride were followed. The titled compound was isolated as a viscous yellow oil (7%).

1.73 N-2-Cyanobenzyl-4-[(N-3-methoxyphenyl)-N-(4-methoxyphenyl)sulphonyl) amino]piperidine

The titled compound was prepared by following the above procedures for de-benzylation (D) of compound 1.34 and then alkylation (E) using 2-cyanobenzyl bromide and isolated as a sticky white solid (51%).

1.74 N-2-Nitrobenzyl-4-[(N-3-methoxyphenyl)-N-(4-methoxyphenyl)sulphonyl) amino]piperidine

The titled compound was prepared by following the above procedures for de-benzylation (D) of compound 1.34 and then alkylation (E) using 2-nitrobenzyl bromide and isolated as a sticky yellow solid (52%).

1.75 N-2-Fluorobenzyl-4-[(N-3-methoxyphenyl)-N-(4-methoxyphenyl)sulphonyl) amino]piperidine

The titled compound was prepared by following the above procedures for de-benzylation (D) of compound 1.34 and then alkylation (E) using 2-fluorobenzyl chloride and isolated as a pale yellow viscous oil (52%).

1.76 N-3-Fluorobenzyl-4-[(N-3-methoxyphenyl)-N-(4-methoxyphenyl)sulphonyl) amino]piperidine

The titled compound was prepared by following the above procedures for de-benzylation (D) of compound 1.34 and then alkylation (E) using 3-fluorobenzyl bromide and isolated as a white viscous oil (24%).

1.77 N-4-Fluorobenzyl-4-[(N-3-methoxyphenyl)-N-(4-methoxyphenyl)sulphonyl) amino]piperidine

The titled compound was prepared by following the above procedures for de-benzylation (D) of compound 1.34 and then alkylation (E) using 4-fluorobenzyl chloride and isolated as a pale yellow viscous oil (50%).

1.78 N-3,5-Difluorobenzyi-4-[(N-3-methoxyphenyl)-N-(4-methoxyphenyl)sulphonyl) amino]piperidine

The titled compound was prepared by following the above procedures for de-benzylation (D) of compound 1.34 and then alkylation (E) using 3,5-difluorobenzyl bromide and isolated as a sticky white solid (40%).

1.79 N-2,6-Dffluorobenzyi-4-[(N-3-methoxyphenyl)-N-(4-methoxyphenyl)sulphonyl) amino]piperidine

The titled compound was prepared by following the above procedures for de-benzylation (D) of compound 1.34 and then alkylation (E) using 2,6-difluorobenzyl bromide and isolated as a viscous white oil (35%).

1.80 N-Cinnamyl-4-[(N-3-methoxyphenyl)-N-(4-methoxyphenyl)sulphonyl) amino]piperidine

The titled compound was prepared by following the above procedures for de-benzylation (D) of compound 1.34 and then alkylation (E) using cinnamyl bromide and isolated as a viscous white oil (17%).

1.81 N-2-Phenylethyl-4-[(N-3-methoxyphenyl)-N-(4-methoxyphenyl)sulphonyl) amino]piperidine

The titled compound was prepared by following the above procedures for de-benzylation (D) of compound 1.34 and then alkylation (E) using phenethyl bromide and isolated as a pale yellow oil (47%).

1.82 N-2-Pyridylmethyl-4-[(N-3-methoxyphenyl)-N-(4-methoxyphenyl)sulphonyl) amino]piperidine

The titled compound was prepared by following the above procedures for de-benzylation (D) of compound 1.34 and then alkylation (E) using 2-picolyl chloride hydrochloride and isolated as a pale yellow viscous oil (35%).

1.83 N-2-Quinolylmethyl-4-[(N-3-methoxyphenyl)-N-(4-methoxyphenyl)sulphonyl) amino]piperidine

The titled compound was prepared by following the above procedures for de-benzylation (D) of compound 1.34 and then alkylation (E) using 2-(chloromethyl)quinoline hydrochloride and isolated as a sticky beige solid (41%).

1.84 N-3-Thienylmethyl4-[(N-3-methoxyphenyl)-N-(4-methoxyphenyl)sulphonyl) amino]piperidine

The titled compound was prepared by following the above procedures for de-benzylation (D) of compound 1.34 and then reductive amination (A) using 3-thiophenecarboxaldehyde and isolated as a sticky white solid (43%).

1.85 N-2-Furylmethyl-4-[(N-3-mothoxyphenyl)-N-(4-methoxyphenyl)sulphonyl) amino]piperidine

The titled compound was prepared by following the above procedures for de-benzylation (D) of compound 1.34 and then reductive amination (A) using 2-furancarboxaldehyde and isolated as a pale yellow viscous oil (45%).

1.86 N-3-pyridylmethyl-4-[(N-3-methoxyphenyl)-N-(4-methoxyphenyl)sulphonyl) amino]piperidine

The titled compound was prepared by following the above procedures for de-benzylation (D) of compound 1.34 and then reductive amination (A) using 3-pyridinecarboxaldehyde and isolated as a pale yellow viscous oil (35%).

1.87 N-2-ethoxybenzyl-4-[(N-3-methoxyphenyl)-N-(4-methoxyphenyl)sulphonyl) amino]piperidlne

The titled compound was prepared by following the above procedures for de-benzylation (D) of compound 1.34 and then reductive amination (A) using 2-4 ethoxybenzaldehyde and isolated as a sticky white solid (37%).

1.88 N-2,3-methylendioxybenzyl-4-[(N-3-methoxyphenyl)-N-(4-methoxyphenyl)sulphonyl)amino]piperidine

The titled compound was prepared by following the above procedures for de-benzylation (D) of compound 1.34 and then reductive amination (A) using 2,3-methylenedioxybenzaldehyde and isolated as a sticky white solid (21%).

1.89 N-2-Methoxybenzyl-4-[(N-3-methoxyphenyl-N-2,5-dimethoxy-4-nitrophenylsulphonyl)amino]piperidine

The above general procedure sulphonylation (C) using N-2-methoxybenzyl4-[(N-3-methoxyphenyl)amino]piperidine and 2,5-dimethoxy-4-nitrobenzenesulphonyl chloride were followed. The titled compound was isolated as a waxy yellow solid (11%).

1.90 N-benzyl-4-[N-(3-(trifluoromethoxy)phenyl)-N-(4-fluorophenylsulphonyl) amino]-piperidine

The above general procedures for reduction amination (A) using 3-(trifluoromethoxy)aniline and 1-benzyl4-piperidone and then sulphonylation (C) using 4-fluorobenzenesulphonyl chloride were followed. The titled compound was isolated as a brown oil (9%).

1.91 N-benzyl-4-[N-(phenyl)-N-(3-methoxyphenylsulphonyl)amino]-piperidine

The above general procedures for reduction amination (A) using aniline and 1-benzyl-4-piperidone and then sulphonylation (C) using 3-methoxybenzenesulphonyl chloride were followed. The titled compound was isolated as a yellow oil (33%).

1.92 N-benzyl-4-[N-(phenyl)-N-(4-(trifluoromethoxy)phenylsulphonyI)amino]-piperidine

The above general procedures for reduction amination (A) using aniline and 1-benzyl-4-piperidone and then sulphonylation (C) using 3-(trifluoromethoxy)benzenesulphonyl chloride were followed. The titled compound was isolated as a white solid (34%).

1.93 N-benzyl-4-[N-(4-methylphenyl)-N-(3-methoxyphenylsulphonyl)amino]-piperidine

The above general procedures for reduction amination (A) using p-toluidine and 1-benzyl-4-piperidone and then sulphonylation (C) using 3-methoxybenzenesulphonyl chloride were followed. The titled compound was isolated as a yellow oil (50%).

1.94 N-benzyl-4-[N-(4-methylphenyl)-N-(4-(trifluoromethoxy)phenylsulphonyl) amino]-piperidine

The above general procedures for reduction amination (A) using p-toluidine and 1-benzyl-4-piperidone and then sulphonylation (C) using 3-(trifluoromethoxy)benzenesulphonyl chloride were followed. The titled compound was isolated as a white solid (55%).

1.95 N-benzyl-4-[N-(4-methylphenyl)-N-(2-naphthylsulphonylamino]-piperidine

The above general procedures for reduction amination (A) using p-toluidine and 1-benzyl4-piperidone and then sulphonylation (C) using 2-naphthalenesulphonyl chloride were followed. The titled compound was isolated as a white solid (56%).

1.96 N-benzyl-4-[N-(3-(trifluoromethoxy)phenyl)-N-(3-methoxyphenylsulphonyl) amino]-piperidine

The above general procedures for reduction amination (A) using 3-(trifluoromethoxy)aniline and 1-benzyl-b 4-piperidone and then sulphonylation (C) using 3-methoxybenzenesulphonyl chloride were followed. The titled compound was isolated as a yellow oil (25%).

1.97 N-benzyl-4-[N-(3-trifluoromethoxy)phenyl)-N-(2-cyanophenylsulphonyl) amino]-piperidine

The above general procedures for reduction amination (A) using 3-(trifluoromethoxy)aniline and 1-benzyl-4-piperidone and then sulphonylation (C) using 2-cyanobenzenesulphonyl chloride were followed. The titled compound was isolated as a colourless oil (12%).

1.98 N-benzyl-4-[N-(3-trifluoromethoxy)phenyl)-N-(3-cyanophenylsulphonyl) amino]-piperidine

The above general procedures for reduction amination (A) using 3-(trifluoromethoxy)aniline and 1-benzyl-4-piperidone and then sulphonylation (C) using 3-cyanobenzenesulphonyl chloride were followed. The titled compound was isolated as a colourless oil (14%).

1.99 N-benzyl4-[N-(3-trifluoromethoxy)phenyl)-N-(2-naphthylsulphonyl)amino]-piperidine

The above general procedures for reduction amination (A) using 3-(trifluoromethoxy)aniline and 1-benzyl4-piperidone and then sulphonylation (C) using 2-naphthalenesulphonyl chloride were followed. The titled compound was isolated as a yellow solid (18%).

1.100 N-(pyridin-4-ylmethyl)-4-[N-(3-trifluoromethoxy)phenyl)-N-(4-methoxyphenylsulphonyl)amino]-piperidine

Following the above procedures for reductive amination (A) using 3-(trifluoromethoxy)aniline and 1-benzyl-4-piperidone and then sulphonylation (C) using 3-(trifluoromethoxy)benzenesulphonyl chloride gave N-benzyl-4-[N-(3-(trifluoromethoxy)phenyl)-N-(4-methoxyphenylsulphonyl)amino]-piperidine. This compound was then subjected to de-benzylation (D) and then reductive amination (A) using 4-pyridinecarboxaldehyde to give the titled compound as a white solid (5%). 1.101 N-(2-chlorobenzyl)-4-[N-(3-trifluoromethoxy)phenyl)-N-(4-methoxyphenylsulphonyl)amino]-piperidine

Following the above procedures for reductive amination (A) using 3-(trifluoromethoxy)aniline and 1-benzyl-4-piperidone and then sulphonylation (C) using 3-(trifluoromethoxy)benzenesulphonyl chloride gave N-benzyl-4-[N-(3-trifluoromethoxyphenyl)-N-(4-methoxyphenylsulphonyl)amino]-piperidine. This compound was then subjected to de-benzylation (D) and then alkylation (E) using 2-chlorobenzyl bromide to give the titled compound as a yellow oil (15%).

1.102 N-benzyl-4-[N-(3-trifluoromethoxy)phenyl)-N-(4-methylphenylsulphonyl) amino]-piperidine

The above general procedures for reduction amination (A) using 3-(trifluoromethoxy)aniline and 1-benzyl-4-piperidone and then sulphonylation (C) using 4-methylbenzenesulphonyl chloride were followed. The titled compound was isolated as a light brown solid (20%).

1.103 N-benzyl-4-[N-(3-trifluoromethoxy)phenyl)-N-(4-tert-butylphenylsulphonyl) amino]-piperidine

The above general procedures for reduction amination (A) using 3-(trifluoromethoxy)aniline and 1-benzyl-4-piperidone and then sulphonylation (C) using 4-tert-butylbenzenesulphonyl chloride were followed. The titled compound was isolated as a light brown solid (18%).

1.105 N-benzyl-4-[(4-t-butylphenyl-N-4-methoxyphenylsulphonyl)amino]piperidine

The above general procedure for reductive amination (A) using 4-t-butylaniline and 1-benzyl4-piperidone and then sulphonylation (C) using 4-methoxybenzenesulphonylchloride were followed. The title compound was isolated as a colourless solid (67.3mg) (25.18%).

1.106 N-benzyi-4-[(4-trifluoromethyl-N-4-methoxyphenylsulphonyl)amino] piperidine

The above general procedure for reductive amination (A) using 4-trifluoromethylaniline and 1-benzyl-4-piperidone and then sulphonylation (C) using 4-methoxybenzenesulphonylchloride were followed. The title compound was isolated as a colourless oil (<9%).

1.107 N-benzyl-4-[(3-trifluoromethyl-N-4- methoxyphenylsulphonyl)amino] piperidine

The above general procedure for reductive amination (A) using 3-trifluoromethylaniline and 1-benzyl4-piperidone and then sulphonylation (C) using 4-methoxybenzenesulphonylchloride were followed. The title compound was isolated as a colourless oil (<9% yield).

1.108 N-benzyl-4-[(4-propylphenyl-N-4- methoxyphenylsulphonyl)amino] piperidine

The above general procedure for reductive amination (A) using 4-propylaniline and 1-benzyl-4-piperidone and then sulphonylation (C) using 4-methoxybenzenesulphonylchloride were followed. The title compound was isolated as a colourless oil 591.9mg (71.2% yield).

1.109 N-benzyl-4[(N-3trifluoromethoxyphenyl-N-4-hydroxyphenylsulphonyl) amino]piperidine

To a solution of compound 1.56 (N-benzyl-4-[(N-3-trifluoromethoxyphenyl)-N-(4-methoxyphenyl)sulphonyl) amino]piperidine) (14mg) in dichloromethane (1 ml) at −78° C. was added Borontribromide (0.05 ml). The cold bath was then removed and the solution allowed to warm to room temperature,while stirring. The reaction was then IS heated to 40° C. and stirred at that temperature overnight. The reaction was then cooled to room temperature and diluted with dichloromethane then washed with two portions of water. The organic extracts were then dryed over Na₂SO₄ and evaporated to dryness. The residue was purified by flash chromatography over silica gel eluted with a gradient of 2% to 4% ammonia/methanol (2M) and dichloromethane, to yield 13.3 mg 97% yeild of the title compound as a yellow oil.

1.110 N-Phenyl-4-[(N-phenyl-N-4-methoxyphenylsulphonyl)amino]piperidine (IV) and 1.111 N-(2-phenethyl)-4-[(N-phenyl-N-(4-methoxyphenyl)sulphonyl)amino] piperidine (V)

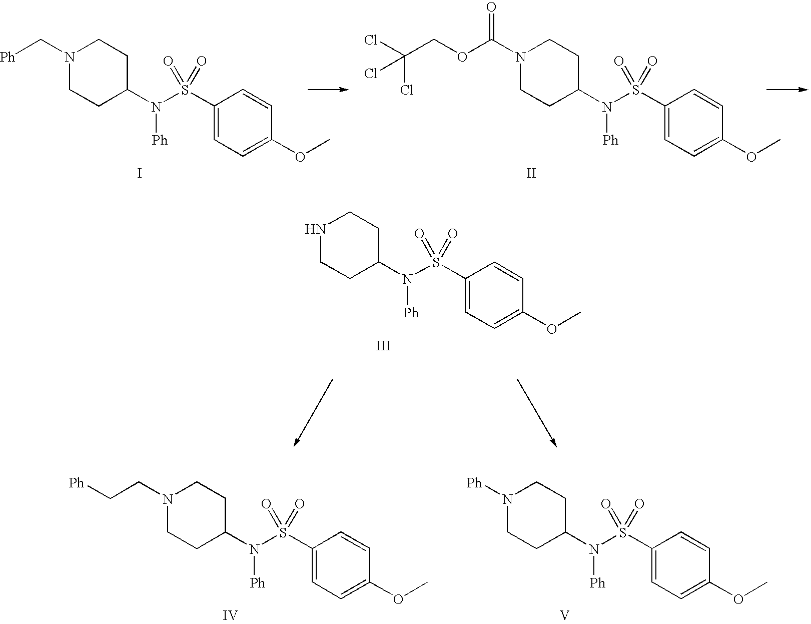

To a solution of benzyl amine I (0.13 mmol) in dichloroethane (2 mL) was added Proton Sponge (0.6 eq) and 2,2,2-trichloroethyl chloroformate (5.0 eq.). After 2 hours the reaction mixture was poured into water and extracted twice with dichloromethane. The organic phase was washed twice with 1 M HCl and once with brine. Drying (sodium sulphate), filtration, and concentration provided a yellow solid. Column chromatography (50% ethyl acetate/hexanes) provided carbamate 11 (83%) as a colourless solid.

To a suspension of carbamate II (0.11 mmol) in methanol (5 mL) was added glacial acetic acid (10 drops) and zinc dust (300 mg). After 1 hour the reaction mixture was filtered. The filtrate was concentrated and the residue was partitioned between ethyl acetate and saturated sodium bicarbonate. The aqueous phase was extracted twice with ethyl acetate. The combined organic phases were washed with-water and brine, dried (sodium sulphate), filtered, and concentrated. Column chromatography (10% methanol/dichloromethane/1% ammonium hydroxide) provided amine 1II (74%) as an off-white semisolid.

To a solution of amine III (0.038 mmol) in anhydrous acetonitrile (2 mL) was added potassium carbonate (10 eq.), potassium iodide (5.0 eq.), and phenethyl bromide (2.0 eq.). The mixture was heated at 90° C. for 18 hours. The cooled reaction mixture was filtered. The filtrate was partitioned between ethyl acetate and water. The organic phase was washed with brine, dried (sodium sulphate), filtered, and concentrated. Column chromatography (2% methanol/dichloromethane/1% ammonium hydroxide) provided amine IV (95%) as an off-white semisolid.

To a solution of amine III (0.038 mmol) in xylenes (2 mL) was added sodium tert-butoxide (1.4 eq.), bromobenzene (4.0 eq.), palladium acetate (trace), and tri(tert-butyl)phosphine (trace). The mixture was heated at 120° C. for 22 hours. The cooled reaction mixture was partitioned between ethyl acetate and water. The organic phase was washed with brine, dried (sodium sulphate), filtered, and concentrated. Column chromatography (25% ethyl acetate/hexanes) provided aniline V (7%) as a colourless oil.

1.112 N-((2-furyl)methyl)-4-[(N-benzyl-N-4-methoxyphenylsulphonyl)amino]piperidine

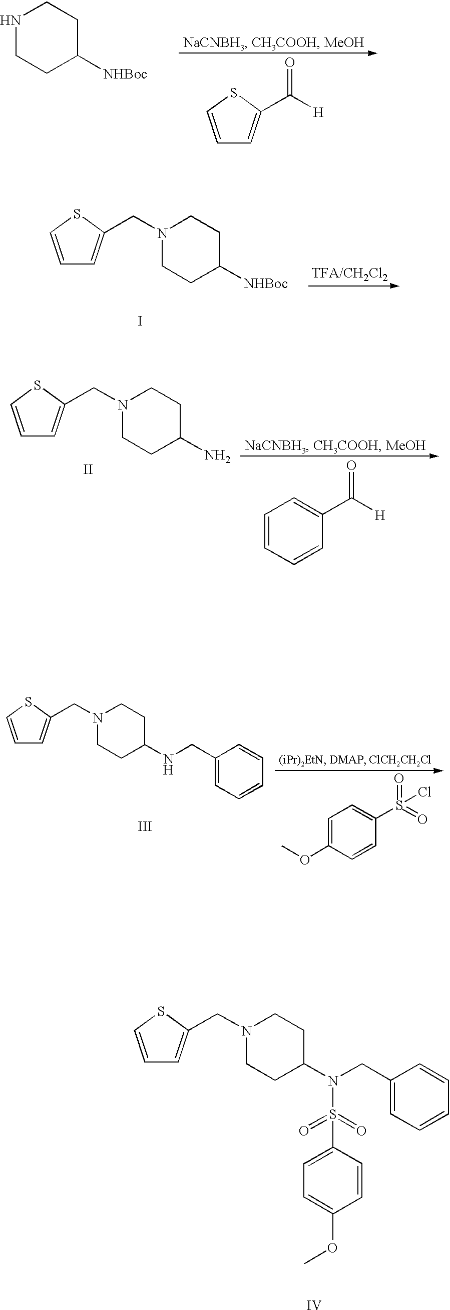

To an ice-cooled solution of 4-N-Boc-amino-piperidine (2.55 mmol) in methanol (6 mL) was added sodium cyanoborohydride (0.6 eq.) and then 2-thiophenecarboxaldehyde (1.1 eq.) and the pH was adjusted to 6 by dropwise addition of acetic acid. The ice bath was removed and the solution was stirred at room temperature for 16 hours. The reaction mixture was then cooled in an ice bath and quenched with a 40% potassium carbonate solution. After extraction with dichloromethane, the combined organic layers were washed with brine, dried over anhydrous sodium sulphate and concentrated. The residue was then purified by flash column chromatography over silica gel eluted with 3% 2M ammonia in methanol/dichloromethane to afford I as a white solid (49%)

The above compound 1 (1.23 mmol) was stirred in a 40% TFA/dichloromethane (2.3 mL) solution for 5 hours. The reaction mixture was then washed with 6N sodium hydroxide and brine, dried over anhydrous sodium sulphate and concentrated to afford II as a yellow oil (100%). The crude material was used directly in the next step.

The same reductive amination procedure was followed as in the first step using the crude material II from the previous step and benzaldeyde. The residue was purified by flash column chromatography over silica gel eluted with 2%-6% 2M ammonia in methanol/dichloromethane to afford III as a brown oil (74%).

To a solution of the amine III (0.12 mmol) in dichloromethane (1 mL) at room temperature was added N,N-diisopropylethylamine (1.5 eq.), 4-methoxybenzenesulphonyl chloride (1.5 eq.), and 4-dimethylaminopyridine (0.15 eq.). The reaction mixture was then stirred at 60 IC for 5 hours. After cooling the mixture was washed with 1 N HCI, water, saturated sodium bicarbonate and brine, dried over anhydrous sodium -sulphate and concentrated. The residue was purified by flash column chromatography over silica gel eluted with 30-40% ethyl acetate/hexane to afford the titled compound IV as a yellow solid (90%).

### EXAMPLE 2

### Solid-Phase Synthesis

A. Michael Addition

A suspension of REM resin (1 g, 1.31 mmol) in N, N-dimethylformamide (15 mL) containing 4-piperidone monohydrate hydrochloride (4 eq.) and N, N-diisopropylethyl-amine (5 eq.) was agitated for 48 hours at room temperature on a shaker. The resin was washed with N, N-dimethylformamide (3×5 mL), dichloromethane (3 x 5 mL), methanol (3×5 mL), and diethyl ether (3×5mL) and dried in vacuo.

B. Reductive Amination

The resin (0.23 mmol) was swollen with a solution of the aniline (10 eq.) and acetic acid (6 eq.) in N, N-dimethylformamide (5 mL). Sodium triacetoxyborohydride (10 eq.) was added as a solid and the suspension was agitated on a shaker for 18 hours at room temperature. The resin was washed with N, N-dimethylfornamide (3×5 mL), dichloromethane (3×5 mL), methanol (3×5 mL), and diethyl ether (3×5 mL) and dried in vacuo.

C. Sulphonylation

The resin (0.23 mmol) was suspended with a solution N, N-diisopropylethylamine (10 to eq.), 4-methoxybenzenesulphonyl chloride (10 eq.), and 4-dimethylaminopyridine (5 eq.) in dichloromethane (5 mL). After agitating on a shaker for 18 hours at room temperature, the resin was washed with N, N-dimethylformamide (3×5 mL), dichloromethane (3×5 mL), methanol (3×5 mL), and diethyl ether (3×5 mL) and dried in vacuo.

D. Quaternization

The resin (0.23 mmol) was swollen with a solution of benzyl bromide (5 eq.) in N, N-dimethylformamide (5 mL) and was agitated on a shaker 18 hours at room temperature. The resin was washed with N, N-dimethylformamide (3×5 mL), dichloromethane (3×5 mL), methanol (3×5 mL), and diethyl ether (3×5 mL) and dried in vacuo.

E. Hofmann Elimination (Cleavage of product from the Resin)

A suspension of the resin (0.23 mmol) in dichloromethane (5 mL) containing N, N-diisopropylethylamine (10 eq.) was agitated on a shaker for 48 hours at room temperature. The resin was drained and washed with dichloromethane (3 x 5 mL). The filtrate was collected and evaporated. The residue was dissolved in dichloromethane and then eluted through an extube using dichloromethane. The extube was pre-treated with a small amount of 1 N sodium hydroxide. The eluent was collected and evaporated and the product was dried in vacuo overnight.

### EXAMPLE 3

### Assay of Transport via GlyT-1

This example illustrates a method for the measurement of glycine uptake by transfected cultured cells.

Cells stably transfected with GlyT-1 C (see Kim, et al., Molecular Pharmacology, 45,1994:608-617) were washed twice with HEPES buffered saline (HBS). The cells were then incubated for 10 minutes at 37° C. with either (a) no potential competitor, (b) 10 mM non-radioactive glycine or (c) a concentration of a candidate drug. A range of concentrations of the candidate drug was used to generate data for calculating the concentration resulting in 50% of the effect (e.g., the IC₅₀, which is the concentration of drug inhibiting glycine uptake by 50%). A solution was then added containing [³H]glycine at a final concentration of 50 nM (17.5 Ci/mmol). The cells were then incubated with gentle shaking for another 30 minutes at 37° C., after which the reaction mixture was aspirated and washed three times with ice-cold HBS. The cells were lysed with scintillant and allowed to equilibrate. The radioactivity in the cells was determined using a scintillation counter. Data was compared between the same cells contacted or not contacted by a candidate drug, depending on the assay being conducted.

The compounds of the present invention were active as GlyT-1 inhibitors.

### EXAMPLE 4

### Assay of Binding to NMDA Receptors-associated Glycine Binding Site

This example illustrates a method used to measure the interaction of compounds to the glycine site on the NMDA receptor. In this assay a known NMDA glycine site binding agent, (tritiated-MDL 105519, available from Amersham), is used to bind to rat hippocampal tissue. The test compound is then introduced and allowed to displace the hot ligand. Binding of the test compound will displace the hot ligand and result in reduced radioactivity, which can be quantified. Compounds are generally tested at two concentrations if inhibition is observed the compounds are retested at several concentrations to generate a dose response curve from which an IC50 may be determined.

The test compounds are prepared for the assay by diluting with 50 mM Tris Acetate buffer. Rat hippocampal membrane aliquots used in the assay are washed twice with cold 10 mM Tris Acetate buffer and subjected to ultracentrifugation at 20,000 rpm for 15 minutes, and rehomogenization between washes. The final pellets are then resuspended in 50 mM Tris Acetate buffer to provide the membranes at a concentration appropriate to the assay. Non-specific binding is defined in the presence of 1 mM glycine. Total binding is defined by the presence of Tris acetate buffer only.

The reaction mixture is prepared by combining 75 μg of homogenized hippocampal membrane preparation with [3H]-MDL 105519 to a final concentration of 5 nM and glycine or test compound as a solution in Tris Acetate Buffer. The reaction is shaken while incubating at room temp for 30 minutes. The plates are then harvested onto GFC filters using a 48 w Brandell Harvestor. The GFC filters are pre-treated for at least 30 minutes with a solution of 0.5% BSA made in distilled water to reduce non-specific binding of the hot ligand to the filter. The plate wells are washed with 4-5 volumes of cold 50 mM Tris Acetate buffer. The filters are then transferred to scintillation vials and 2 mls of scintillant is added to each vial. The vials are allowed to sit overnight before being counted in a Beckman β-counter. The data is analyzed using Prism software. The compounds of the present invention show no significant binding to the NMDA receptor-associated glycine binding site.

### EXAMPLE 5

### Glycine Receptor Binding Assay

This example illustrates an assay used to measure cross reactivity of the compounds with the Glycine receptor. In this assay the known glycine receptor binding agent, [3H]-Strychnine is used to bind to rat spinal cord tissue. The test compound is then introduced and allowed to displace the hot ligand. Binding of the test compound will displace the hot ligand and result in reduced radioactivity, which can be quantified. Compounds are generally tested at two concentrations, if inhibition is observed the compounds are retested at several concentrations to generate a dose response curve from which an IC50 may be determined.

The test compounds are prepared for the assay by diluting in potassium phosphate buffer. The aliquots of rat spinal cord membrane used in the assay are washed with two portions of cold Phosphate buffer followed by microcentrifugation at 4° C., at 14,000 rpm between washings. The final pellets are then resuspended in a volume of phosphate buffer to provide concentrations appropriate to the assay conditions. The non-specific and total binding are defined by 10 mM final concentration of glycine and phosphate buffer only, respectively.

The reaction mixture is prepared by combining 150 μg of the rat spinal cord membrane with [3H]-strychnine to a final concentration of 7 nM and glycine or test compound. The reaction mixture is incubated for two hours while shaking on ice. The plates are then harvested onto GFC filters using a 48 w Brandall Harvestor. The GFC filter is pre-reated for at least 30 minutes with a solution of 0.5% BSA made is distilled water to reduce non-specific binding. The plate wells are washed with 4-5 volumes of cold phosphate buffer. The filters are then transferred to scintillation vials and 2mls of scintillant is added to each vial. The vials are allowed to sit overnight before being counted in a Beckman β-counter. The data is analyzed using Prism software.

The compounds of the present invention show no significant binding to the glycine receptor.

| Experiment No. | Structure |
|---|---|
| 1.63 | 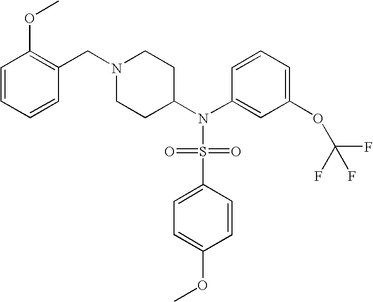 |
| 1.75 | 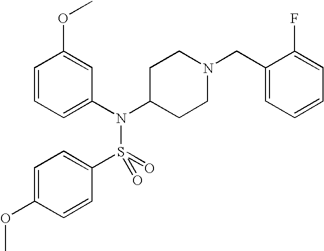 |
| 1.64 | 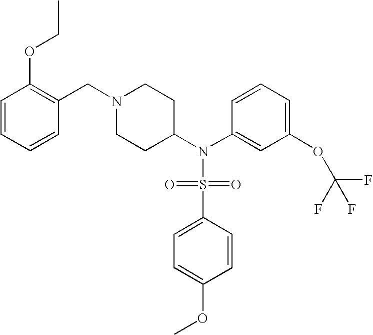 |
| 1.87 | 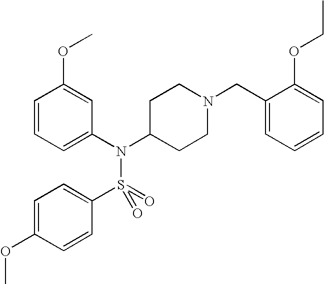 |
| 1.49 | 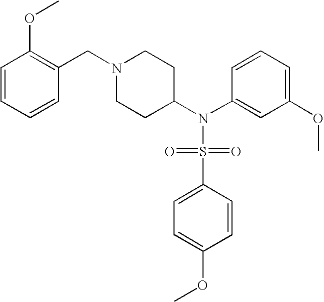 |
| 1.58 | 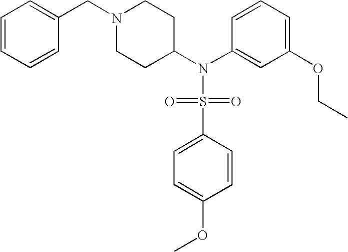 |
| 1.88 | 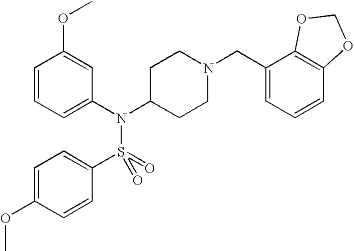 |
| 1.48 | 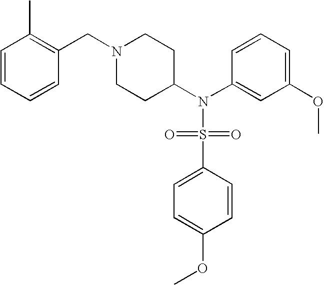 |
| 1.84 | 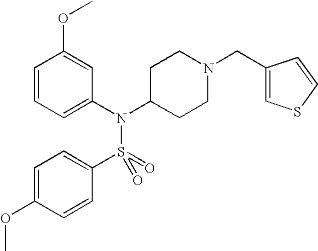 |
| 1.56 | 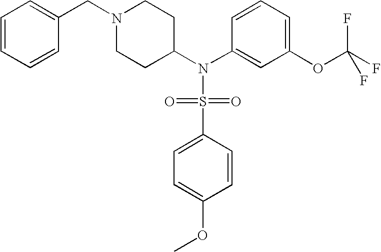 |
| 1.77 | 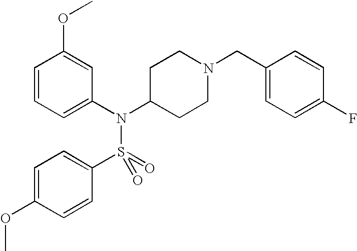 |
| 1.34 | 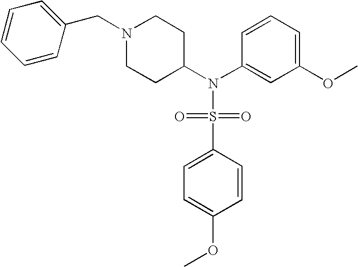 |
| 1.73 | 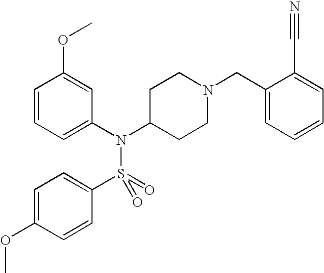 |
| 1.35 | 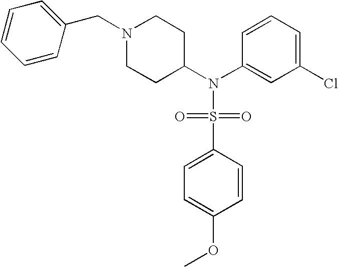 |
| 1.36 | 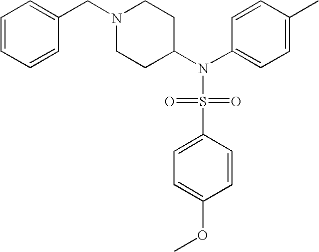 |
| 1.55 | 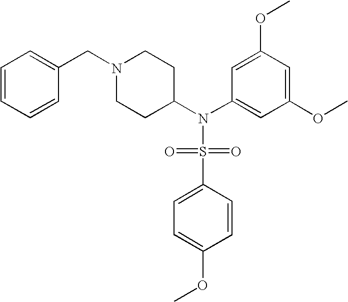 |
| 1.86 | 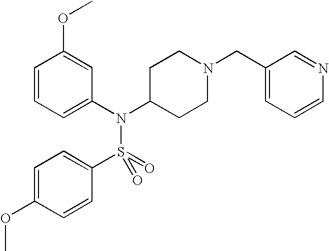 |
| 1.106 | 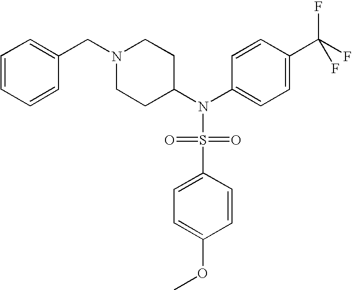 |
| 1.108 | 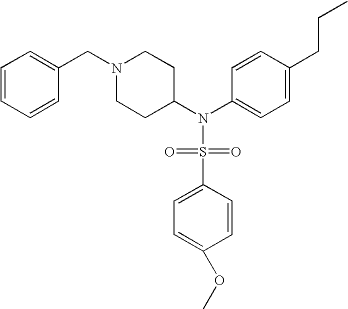 |
| 1.38 | 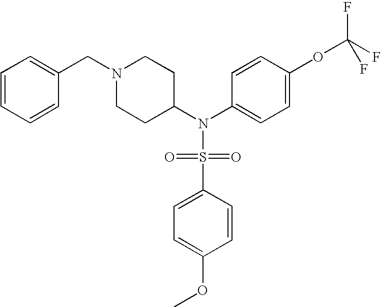 |
| 1.62 | 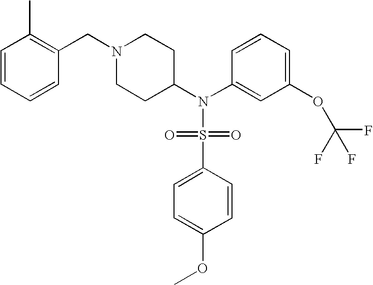 |
| 1.76 | 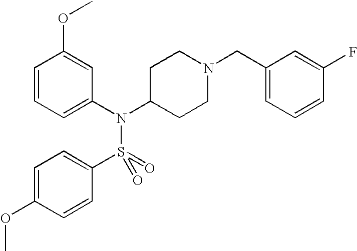 |
| 1.90 | 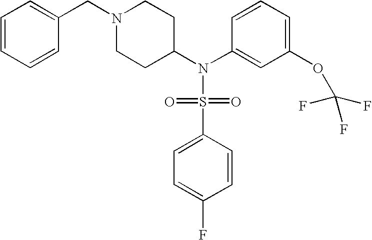 |
| 1.33 | 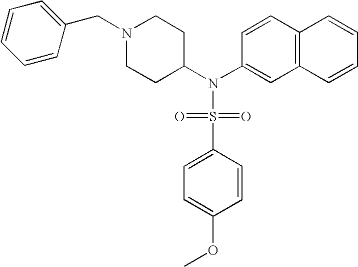 |
| 1.37 | 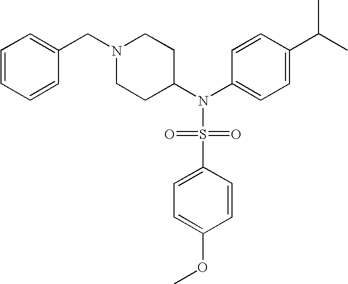 |
| 1.82 | 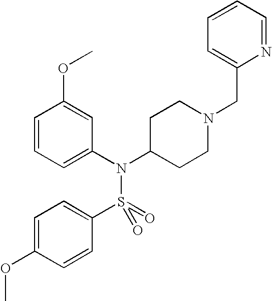 |
| 1.102 | 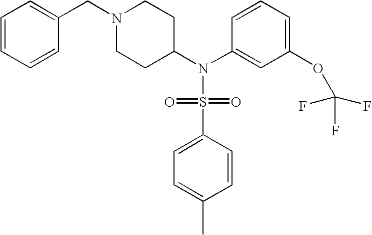 |
| 1.59 | 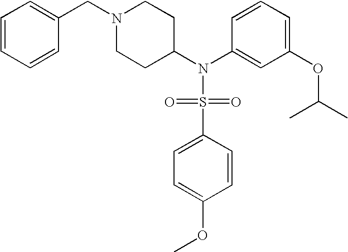 |
| 1.4 | 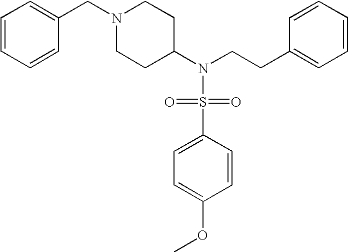 |
| 1.107 | 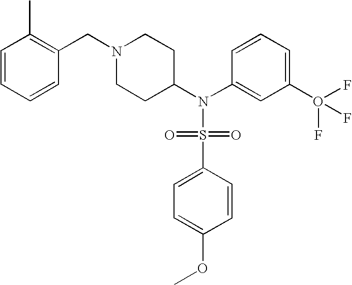 |
| 1.74 | 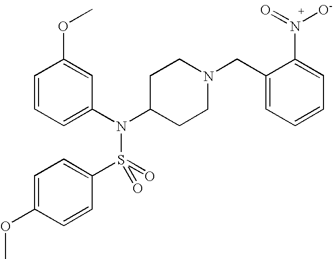 |
| 1.31 | 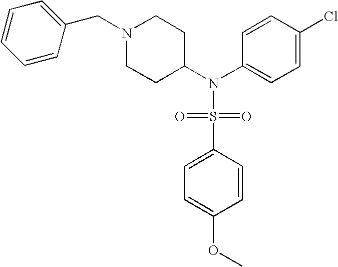 |
| 1.109 | 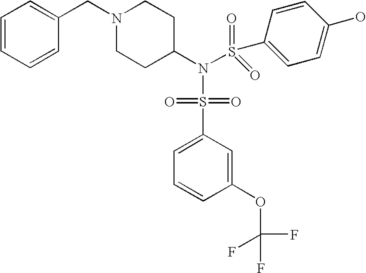 |
| 1.100 | 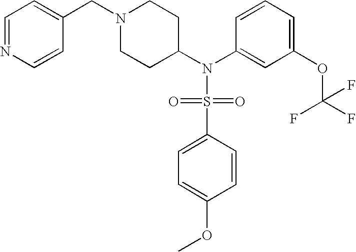 |
| 1.79 | 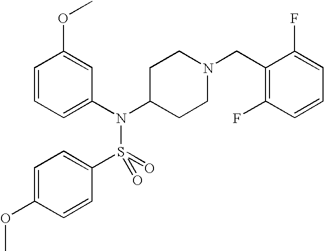 |
| 1.96 | 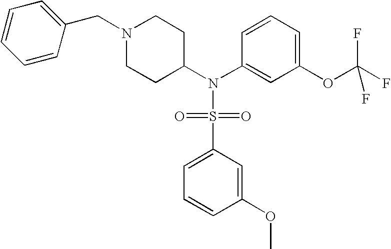 |
| 1.66 | 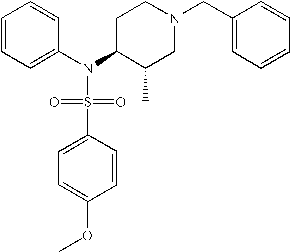 |
| 1.3 | 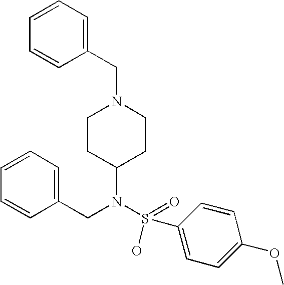 |
| 1.78 | 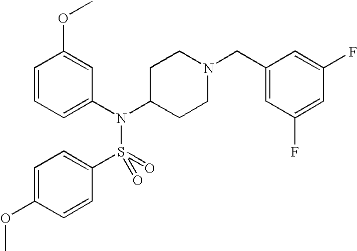 |
| 1.45 | 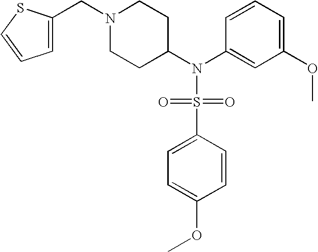 |
| 1.16 | 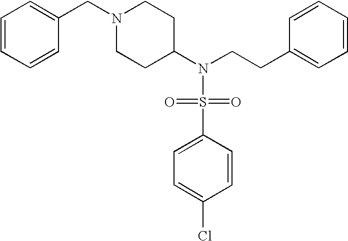 |
| 1.103 | 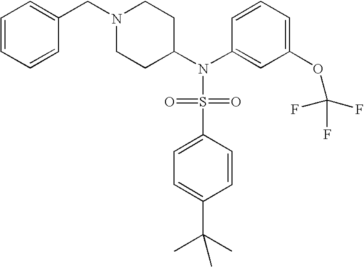 |
| 1.97 | 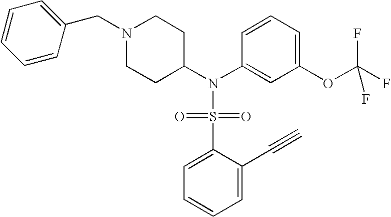 |
| 1.22 | 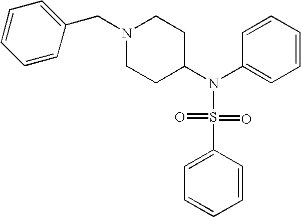 |
| 1.71 | 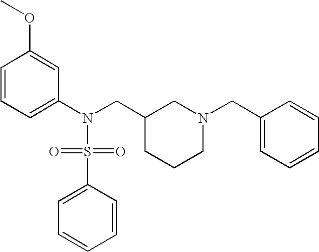 |
| 1.105 | 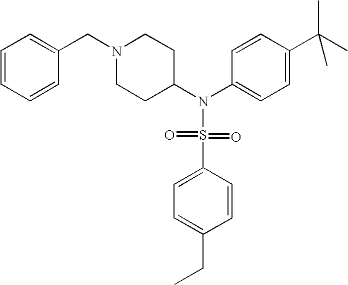 |
| 1.112 | 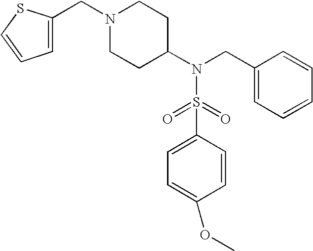 |
| 1.41 | 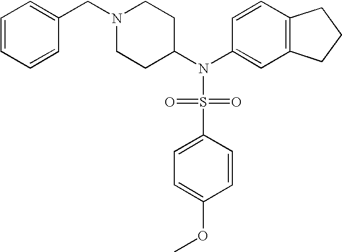 |
| 1.40 | 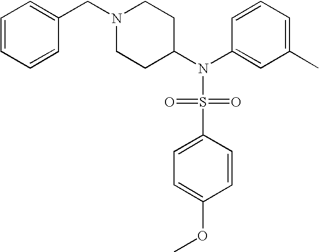 |
| 1.51 | 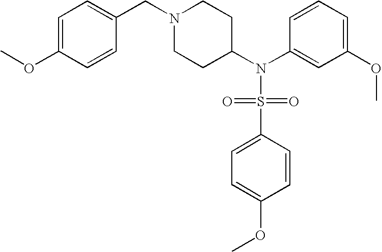 |
| 1.101 | 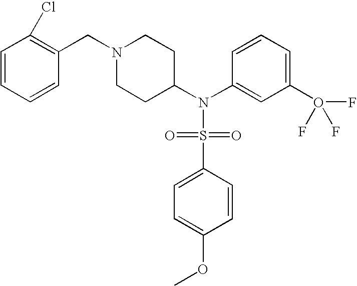 |
| 1.50 | 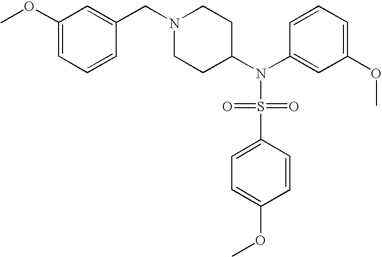 |
| 1.9 | 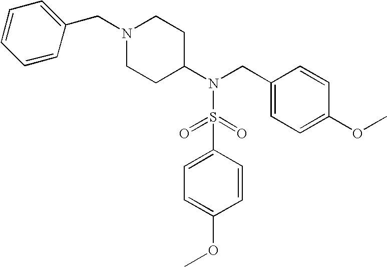 |
| 1.85 | 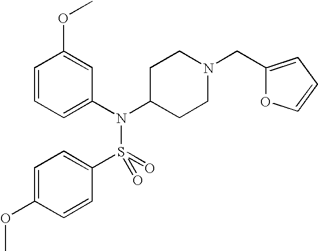 |
| 1.8 | 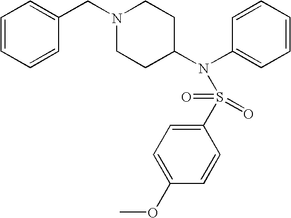 |
| 1.47 | 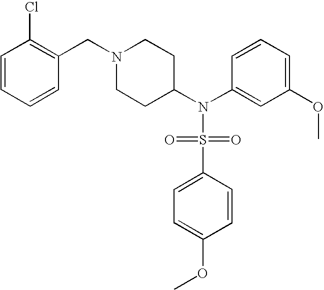 |
| 1.53 | 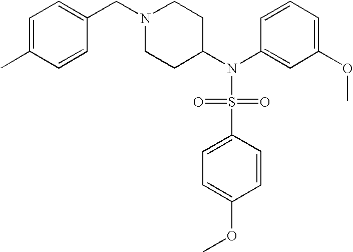 |
| 1.54 | 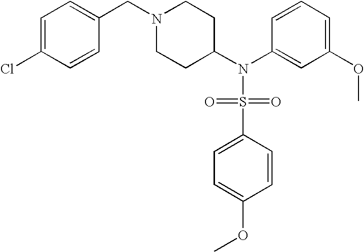 |
| 1.30 | 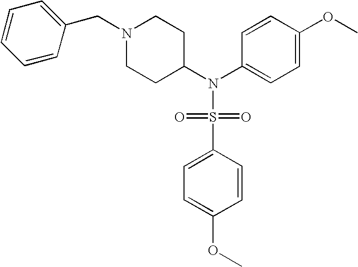 |
| 1.94 | 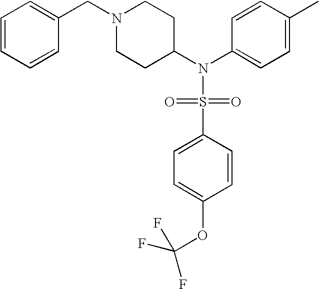 |
| 1.21 | 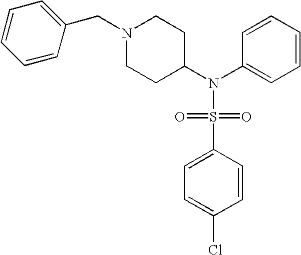 |
| 1.52 | 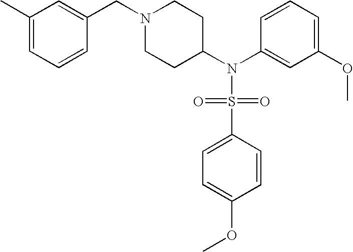 |
| 1.17 | 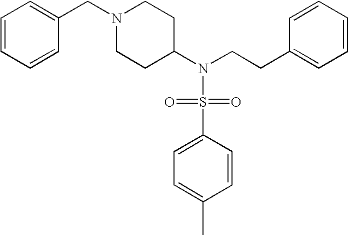 |
| 1.93 | 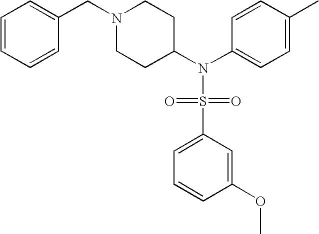 |
| 1.99 | 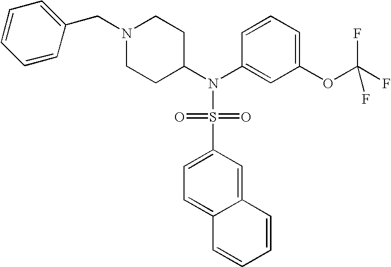 |
| 1.2 | 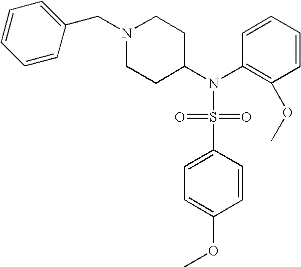 |
| 1.83 | 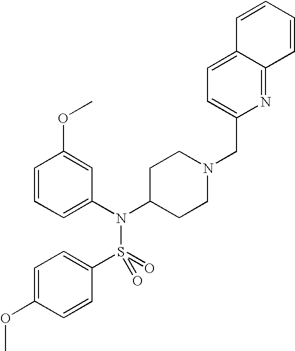 |
| 1.43 | 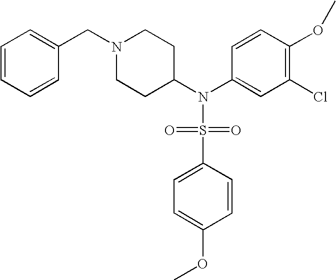 |
| 1.95 | 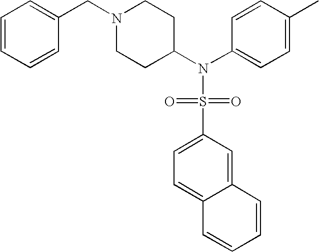 |
| 1.67 | 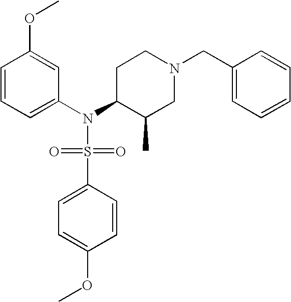 |
| 1.44 | 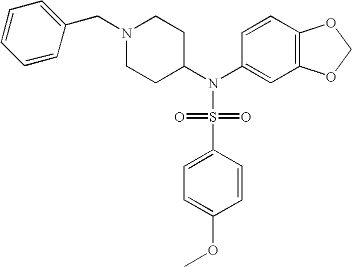 |
| 1.80 | 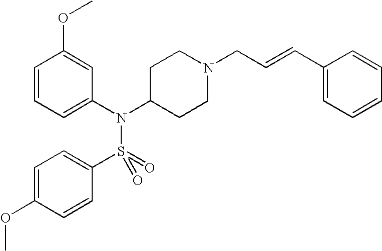 |
| 1.42 | 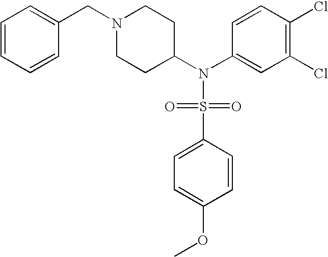 |
| 1.15 | 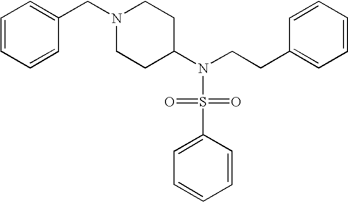 |
| 1.81 | 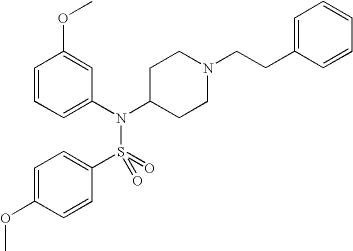 |
| 1.46 | 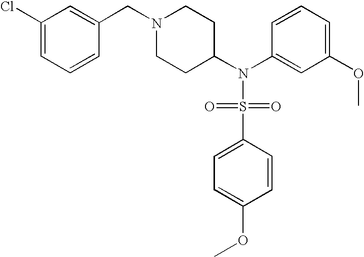 |
| 1.91 | 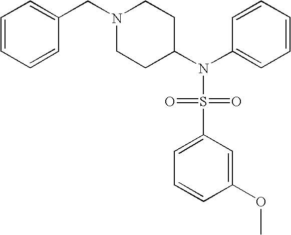 |
| 1.57 | 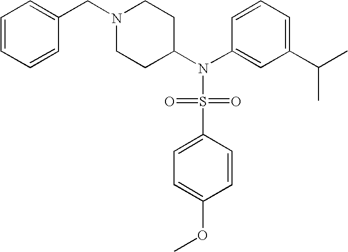 |
| 1.11 | 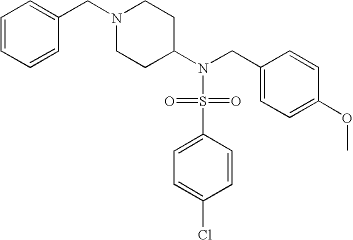 |
| 1.92 | 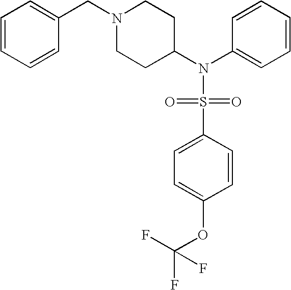 |
| 1.19 | 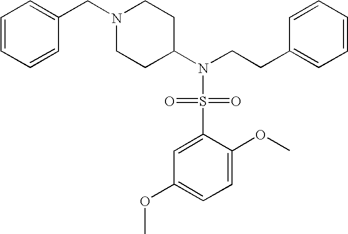 |
| 1.89 | 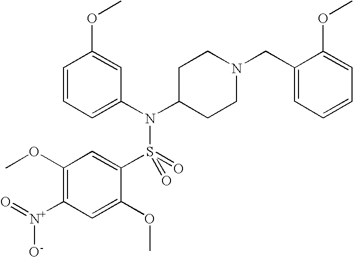 |
| 1.98 | 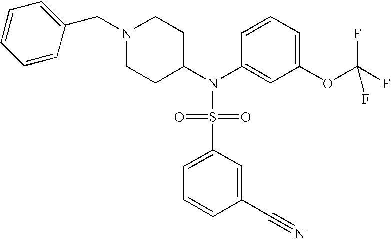 |
| 1.29 | 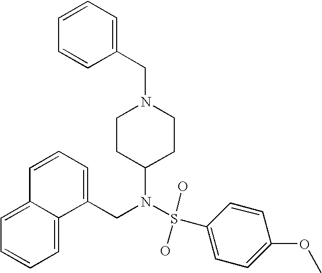 |
| 1.25 | 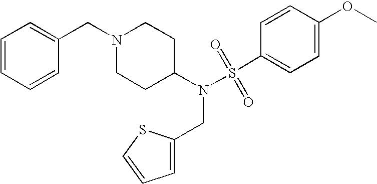 |
| 1.26 | 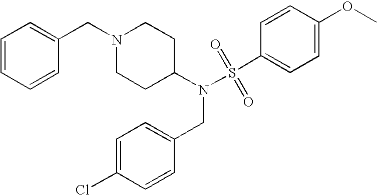 |
| 1.70 | 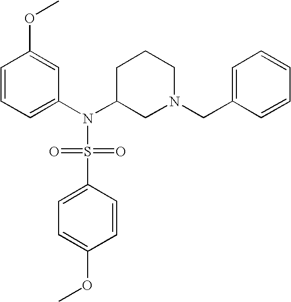 |
| 1.72 | 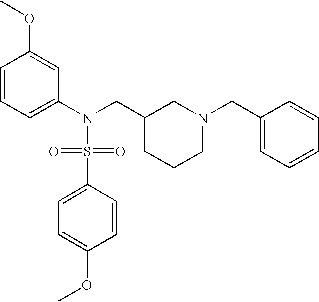 |
| 1.60 | 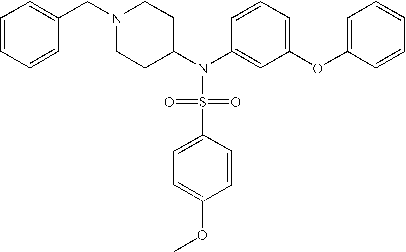 |
| 1.39 | 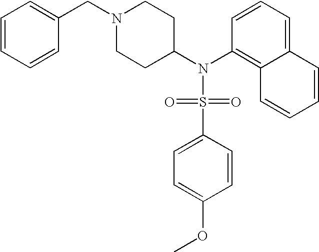 |
| 1.32 | 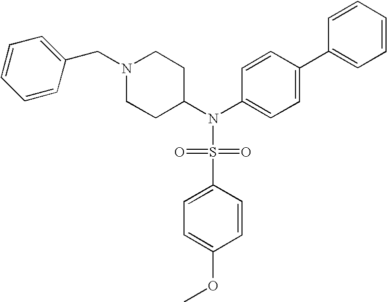 |
| 1.24 | 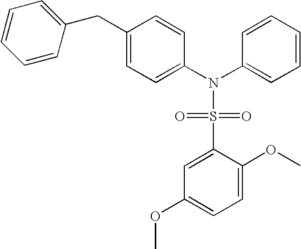 |
| 1.23 | 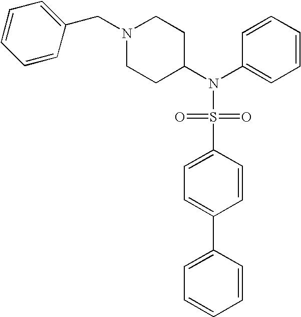 |
| 1.20 | 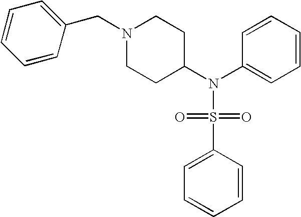 |
| 1.18 | 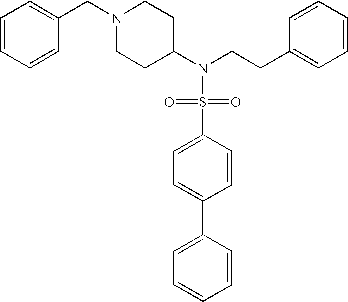 |
| 1.14 | 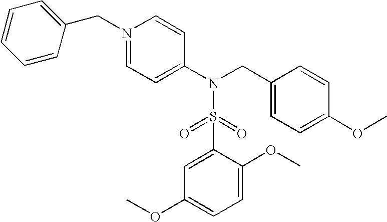 |
| 1.13 | 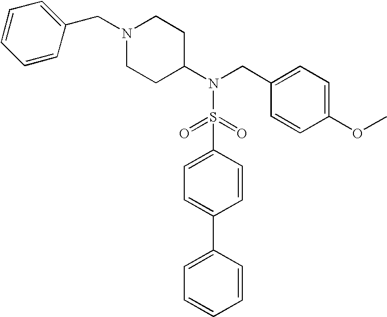 |
| 1.12 | 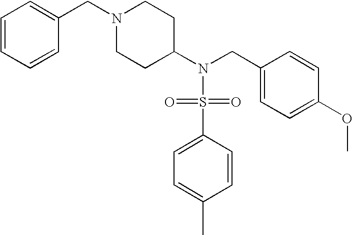 |
| 1.10 | 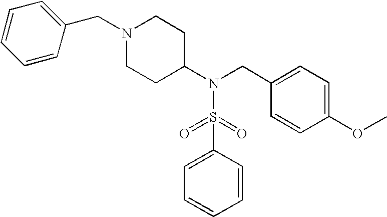 |
| 1.65 | 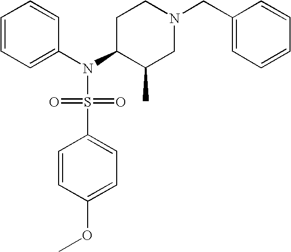 |
| 1.27 | 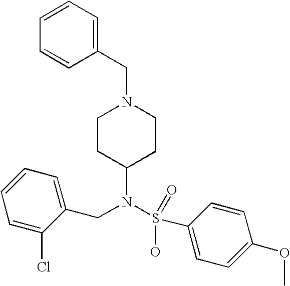 |
| 1.5 | 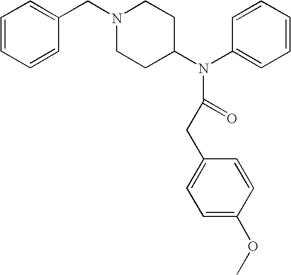 |
| 1.7 | 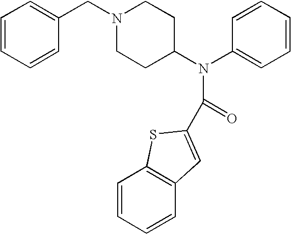 |
| 1.28 | 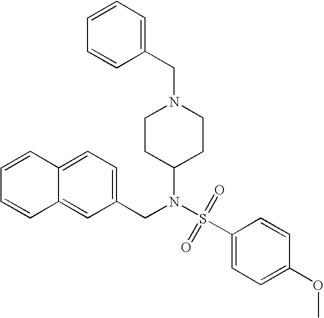 |
| 1.6 | 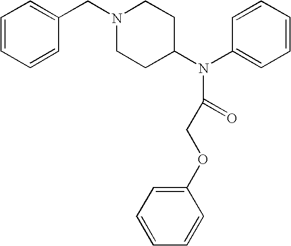 |
| 1.30 | 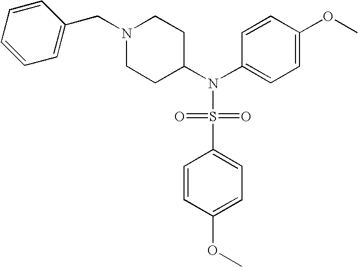 |

We claim:

1. A compound according to Formula I: 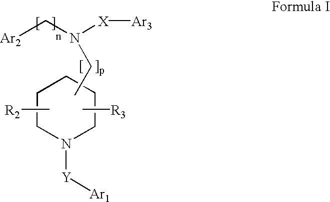
wherein:
X is a C=O group or an SO₂ group;
Y is selected from the group consisting of —[CHR₁]ₘ-, -CH₂CH≡CH-, and CH₂C=-C-,
m is an integer selected from the group consisting of 1, 2, and 3;
n is an integer selected from the group consisting of 0, 1, and 2;
p is an integer selected from the group consisting of 0, 1, and 2;
R₁, R₂ and R₃ are independently selected from the group consisting of H and alkyl;
Ar₁, Ar₂ and Ar₃ are independently selected aryl groups, optionally substituted with up to five substituents independently selected from the group consisting of alkyl, alkoxy, cycloalkyl, cycloalkyloxy, heterocycloalkyl, heterocycloalkyloxy, alkanoyl, thioalkyl, aralkyl, aralkoxy, aryloxyalkyl, aryloxyalkoxy, cycloalkyl-substituted alkyl, cycloalkyloxy-substituted alkyl, cycloalkyl-substituted alkoxy, cycloalkyloxy-substituted alkoxy, heterocycloalkyl-substituted alkyl, heterocycloalkyloxy-substituted alkyl heterocycloalkyl-substituted alkoxy, heterocycloalkyloxy-substituted alkoxy, thioaryl, aralkylthio, thioaryl-alky, halo, NO₂, Ph, CF₃, OCF₃, CN, OH, methylenedioxy, ethylenedioxy, SO₂NRR′, NRR′, CO₂R (where R and R′ are independently selected from the group consisting of H and alkyl) and aryl. The aryl substituent may be further substituted with any substituent selected from the preceding list;
with the following provisos:
(iii) when X is a C=O group, n is 0 and Ar₁ is not a 2,3-dihydro-ibdol-5-yl or 2,3w dihydro-indol-6-yl group;
(iv) when X is an S0₂ group and n is 0, Ar₂ is not a 2,3-dihydro-indol-5-yl or 2,3-dihydro-indol-6-yl group;
and a salt, solvate and hydrate thereof.

2. A compound according to claim 1 wherein X is SO₂.

3. A compound according to claim 2 wherein m is selected from the group consisting of 1 and 3;

4. A compound according to 3 wherein R₁ and R3 are each H and R₂ is methyl.

5. A compound according to claim 3 wherein R₁, R₂ and R₃ are each H.

6. A compound according to claim 5 wherein Y is [CHR₁]-ₘ, m is 2 and R1 is H and Ar₁ is phenyl,

7. A compound according to claim 5 wherein Y is [CHR₁]-ₘ, m is 0 and Ar₁ is phenyl.

8. A compound according to claim 5 wherein Y is [CHR₁]ₘ, m is 1 and R₁ is H.

9. A compound according to claim 8 wherein the amino substituent attaches at the 3 position of the piperidine ring and p is 1.

10. A compound according to claim 8 wherein the amino substituent attaches at the 4 position of the piperidine ring.

11. A compound according to claim 10 wherein p is 0.

12. A compound according to claim 11 wherein n is 1 and Ar₂ is selected from the group consisting of phenyl and naphthyl.

13. A compound according to claim 11 wherein n is 2 and Ar₂ is phenyl.

14. A compound according to claim 11 wherein n is 0.

15. A compound according to claim 14 wherein Ar3 is selected from phenyl or naphthyl and is optionally substituted with one or more substituents selected from the group consisting of alkyl, alkoxy, cycloalkyl, cycloalkyloxy, heterocycloalkyl, heterocycloalkyloxy, alkanoyl, thioalkyl, aralkyl, aralkoxy, aryloxyalkyl, aryloxyalkoxy, cycloalkyl-substituted alkyl, cycloalkyloxy-substituted alkyl, cycloalkyl-substituted alkoxy, cycloalkyloxy-substituted alkoxy, heterocycloalkyl-substituted alkyl, heterocycloalkyloxy-substituted alkyl heterocycloalkyl-substituted alkoxy, heterocycloalkyloxy-substituted alkoxy, thioaryl, aralkylthio, thioaryl-alky, halo, NO₂, Ph, CF₃, OCF₃, CN, OH, methylenedioxy, ethylenedioxy, SO₂NRR′, NRR′, CO₂R (where R and R′ are independently selected from the group consisting of H and alkyl) and aryl. The aryl substituent may be further substituted with any substituent selected from the preceding list;

16. A compound of claim 15 wherein Ar₃ is optionally substituted phenyl wherein the substituent is selected from the group consisting of alkyl, alkoxy, halo, CN, CF₃, OCF₃, and OH.

17. A compound of claim 16 wherein Ar₁ is selected from optionally substituted phenyl, pyridine, thiophenyl, furan, quinoline optionally substituted with one or more substituents selected from the group consisting of alkyl, alkoxy, cycloalkyl, cycloalkyloxy, heterocycloalkyl, heterocycloalkyloxy, alkanoyl, thioalkyl, aralkyl, aralkoxy, aryloxyalkyl, aryloxyalkoxy, cycloalkyl-substituted alkyl, cycloalkyloxy-substituted alkyl, cycloalkyl-substituted alkoxy, cycloalkyloxy-substituted alkoxy, heterocycloalkyl-substituted alkyl, heterocycloalkyloxy-substituted alkyl heterocycloalkyl-substituted alkoxy, heterocyc(oalkyloxy-substituted alkoxy, thioaryl, aralkylthio, thioaryl-alky, halo, NO₂, Ph, CF₃, OCF₃, CN, OH, methylenedioxy, ethylenedioxy, SO₂N RR′, NRR′, CO₂R (where R and R′ are independently selected from the group consisting of H and alkyl) and aryl. The aryl substituent may be further substituted with any substituent selected from the preceding list.

18. A compound of according to claim 17 wherein Ar₁ is optionally susbtituted phenyl wherein one or more substituents are selected from the group consisting of alkyl, alkoxy, cycloalkyl, cycloalkyloxy, heterocycloalkyl, heterocycloalkyloxy, alkanoyl, thioalkyl, aralkyl, aralkoxy, aryloxyalkyl, aryloxyalkoxy, cycloalkyl-substituted alkyl, cycloalkyloxy-substituted alkyl, cycloalkyl-substituted alkoxy, cycloalkyloxy-substituted alkoxy, heterocycloalkyl-substituted alkyl, heterocycloalkyloxy-substituted alkyl heterocycloalkyl-substituted alkoxy, heterocycloalkyloxy-substituted alkoxy, thioaryl, aralkylthio, thioaryl-alky, halo, NO₂, Ph, CF₃, OCF₃, CN, OH, methylenedioxy, ethylenedioxy, SO₂NRR′, NRR′, CO₂R (where R and R′ are independently selected from the group consisting of H and alkyl) and aryl.
The aryl substituent may be further substituted with any substituent selected form the preceding list.

19. A compound according to claim 18 wherein Ar₁ is phenyl.

20. A compound according to claim 18 wherein Ar₁ is optionally substituted phenyl wherein one or more substituents are selected from the group consisting of alkyl, alkoxy, halo, methylendioxy, NO₂, and CN.

21. A compound according to claim 20 wherein Ar₁ is phenyl optionally substituted with one or two substituents selected from, Cl, F, Me, methoxy, and ethoxy.

22. A compound according to claim 21 wherein Ar₁ is monosubstitueted phenyl and the substituent is located at the 2-position.

23. A compound according to claim 17 wherein Ar₂ is optionally substituted with one or more substituents and is selected from the group consisting of phenyl, thiophene, naphthalene and indane, wherein the substituents are selected from the group consisting of alkyl, alkoxy, cycloalkyl, cycloalkyloxy, heterocycloalkyl, heterocycloalkyloxy, alkanoyl, thioalkyl, aralkyl, aralkoxy, aryloxyalkyl, aryloxyalkoxy, cycloalkyl-substituted alkyl, cycloalkyloxy-substituted alkyl, cycloalkyl-substituted alkoxy, cycloalkyloxy-substituted alkoxy, heterocycloalkyl-substituted alkyl, heterocycloalkyloxy-substituted alkyl heterocycloalkyl-substituted alkoxy, heterocycloalkyloxy-substituted alkoxy, thioaryl, aralkylthio, thioaryl-alky, halo, NO₂, Ph, CF₃, OCF₃, CN, OH, methylenedioxy, ethylenedioxy, SO₂NRR′, NRR′, CO₂R (where R and R′ are independently selected from the group consisting of H and alkyl) and aryl. The aryl substituent may be further substituted with any substituent from the preceding list.

24. A compound according to claim 23 wherein Ar₂ is selected from optionally substituted phenyl wherein one or more substituents are selected from the group consisting of alkyl, alkoxy, halo, CF₃, and OCF₃.

25. A compound according to claim 24 wherein Ar₂ is phenyl substituted at the 3-position.

26. A compound selected from the group consisting of:
N-Benzoyl-4-[(N-benzyl-N-4-methoxyphenylsulphonyl)amino]-piperidine (1.1);
N-Phenyl-4-[(N-phenyl-N-4-methoxyphenylsulphonyl)amino]-piperidine (1. 11 0);
N-Benzyl-4-[(N-2-methoxyphenyl-N-4-methoxyphenylsulphonylamino]-piperidine (1.2);
N-Benzyl-4-[(N-benzyl-N-4-methoxyphenylsulphonyl)amino]-piperidine (1.3);
N-Benzyl-4-[(N-2-phenylethyl-N-(4-methoxyphenylsulphonyl)amino]-piperidine (1.4);
N-Benzyl-4-[(N-phenyl)-N-(4-methoxyphenyl)acetyl)amino]-piperidine (1.5);
N-Benzyl-4-[(N-phenyl)-N-(phenoxy)acetyl)amin6]-piperidine (1.6);
N-Benzyl-4-[(N-4-methoxybenzyl-N-4-methoxyphenylsulphonyl)amino]-piperidine (1.9);
N-Benzyl-4-f(N-phenylJ-N-((2-benzo[b]thiophenyl)carbonyl)amino]-piperidine (1.7);
N-Benzyl-4-[(N-phenyl-N-4-methoxyphenylsulphonyl)amino]-piperidine (1.8);
N-Benzyl-4-[(N-4-methoxybenzyl-N-phenylsulphonyl)amino]-piperidine (1.10);
N-Benzyl-4-[(N4-methoxybenzyi-N-4-chlorophenylsulphonylamino]-piperidine (1.11);
N-Benzyl-4-[(N-4-methoxybenzyl-N-4-methylphenylsulphonyl)amino]-piperidine (1.12);
N-Benzyl-4-[(N-4-methoxybenzyl-N-4-pheny/phenylsulphonyl)amino]-piperidine (1.13);
N-Benzyl-4-[(N4-methoxybenzyI-N-2, 5-dimethoxyphenylsulphonyl)amino]-piperidine (1.14);
N-Benzyl-4-[(N-2-phenylethyl-N-phenylsulphonyl)amino]-piperidine (1.15);
N-Benzyl-4-[(N-2-phenylethyl-N-(4-chloropheny11sulphonyl)amino;-pipridine (1.16);
N-Benzyl4-[(N-2-phenylethyl-N-(4-methylphenyl)sulph;nyl)amino]piperidine (1.17);
N-Benzyl-4-[(N-2-phenylethyl-N-(4-phenylphenyl)sulphonyl)amino]-piperidine (1.18);
N-Benzyl-4-[(N-2-phenylethyl-N-(2, 5-dimethoxyphenyl)sulphonyl)amino]-piperidine (1.19);
N-Benzyl4-[(N-phenyl-N-phenylsulphonyl)amino]-piperidine (1.20);
N-Benzyl-4-[(N-phenyl-N-(4-chlorophenyl)sulphonyl)amino]-piperidine (1.21);
N-Benzyl-4-[(N-phenyl-N-(4-methylphenyl)sulphonyl)amino]-piperidine (1.22);
N-Benzyl-4-[(N-phenyl-N-(4-phenylphenyl)sulphonyl)amino]-piperidine (1.23);
N-Benzyl-4-[(N-phenyl-N-(2,5-dimethoxyphenyl)sulphonyl)amino]-piperidine (1.24);
N-((2-fury)methyl)-4-[(N-benzyl-N-4-methoxyphenylsulphonyl)amino]-piperidine (1.1112);
N-Benzyl-4-[(N-((2-fur)methylJ-N-(4-methoxyphenyl)sulphonyl)amino]-piperidine (1.25);
N-Benzyl4-[(N-(4-chlorobenzyl)-N-(4-methoxyphenyl)sulphonyl)amino]-piperidine (1.26);
N-Benzyl-4-[(N-2-chlorobenzyl)-N-(4-methoxyphenyl)sulphonyl)amino]piperidine (1.27);
N-Benzyl4-[(N-(2-naphthyl)methyl)-N-(4-methoxyphenyl)sulphonyl)amino]-piperidine (1.28);
N-Benzyl-4-[(N-1 -naphthylmethyl)-N-(4-methoxyphenyl)sulphonyl)amino]-piperidine (1.29);
N-Benzyl4-[(N-(4-methoxypheny )-N-(4-methoxyphenyl)sulphonyl)amino]-piperidine (1.30);
N-Benzyl4-[(N-4-chlorophanyl)-N-(4-methoxyphenyf)sutphonyl)amino]-piperidine (1.31);
N-(2-phenethyl)-4-[(Nphenyl-N-(4-methoxyphenyl)sulphonyf)amino]-piperidine (1.111);
N-Benzyl-4-[(N-4-phenylphenyl)-N-(4-methoxyphenyl)sulphonyl)amino]-piperidine (1.32);
N-Benzyl-4-[(N-(2-naphthyl)-N-(4-methoxyphenyl)sulphonyl)amino]-piperidine (1.33);
N-Benzyl-4-[(N-3-methoxyphenyl)-N-(4-methoxyphenyl)sulphonyl)amino]-piperidine (1.34);
N-Benzyl-4-[(N-3-chlorophenyl)-N-(4-methoxyphenyl)sulphonyl)amino]-piperidine (1.35);
N-Benzyl-4-[(N-4-methylphenylJ-N-(4-methoxyphenyl)sulphonyl)amino]-piperidine (1.36);
N-Benzyl-4-[(N-4-isopropylphenyl)-N-(4-methoxyphenyl)sulphonyl)amino]piperidine (1.37);
N-Benzyl-4-[(N-4-trifluoromethoxyphenyl)-N-(4-methoxyphenyl)sulphonyl)amino]-piperidine (1.38);
N-Benzyl-4-[(N-(I-naphthyl))-N-(4-methoxyphenyfsutphonyl)amino]-piperdine (1.39);
N-Benzyl-4-[(N-3-methylphenyl)-N-(4-methoxyphenyl)suiphonyl)amino]-piperidine (1.40);
N-Benzyl-4-[(N-(5-indanyl))-N-(4-methoxyphenyl)sulphonyl)amino]-piperidine (1.41);
N-Benzyl-4-[(N-3, 4-dichlorophenyl)-N-(4-methoxyphenyl)sulphonyl)amino]-piperidine (1.42);
N-Benzyl-4-[(N-3-chloro-4-methoxyphenyl)-N-(4-methoxyphenyl)sulphonyl)amino]-piperidine (1.43);
N-Benzyl-4-[(N-3, 4-methylendioxyphenyl)-N-(4-methoxyphenyl)sulphonyl)amino]-piperidine (1.44);
N-(2-furylmethyl)-4-[(N-3-mothoxyphenyl)-N-(4-mothoxyphenylpsulphonylpamino]-piperidine (1.45);
N-3-chlorobenzyl-4-[(N-3-methoxyphenyl-N-(4-methoxyphenyl)sulphonyl)amino]-piperidine (1.46);
N-(2-Chlorobenzyl)-4-[(N-3-methoxybenzyl)-N-(4-methoxyphenyI)sulphonyl)amino]-piperidine (1.47);
N-2-methylbenzyl-4-[(N-3-methoxyphenyl)-N-(4-methoxyphenyl)sulphonyl)amino]-piperidine (1.48);
N-2-methoxybenzyl-4-[(N-3-methoxyphenyl)-N-(4-methoxyphenyl)sulphonyl)amino]-piperidine (1.49);
N-3-methoxybenzyl-4-[(N-3-methoxyphenyl)-N-(4-methoxyphenyl)sulphonyl)amino]-piperidine (1.50);
N-4-methoxybenzyl-4-[(N-3-methoxyphenyl)-N-(4-methoxyphenyl)suiphonyluamina]-piperidine (1.51);
N-3-methylbenzyl-4-[(N-3-methoxyphenyl)-N-(4-methoxyphenyl)sulphonyl)amino]-piperidine (1.52);
N4-methylbenzyl-4-[(N-3-methoxyphenyl)-N-(4-methoxyphenyl)sulphonyl)amino]-piperidine (1.53);
N-4-chlorobenzyl-4-[(N-3-methoxyphenyl)-N-(4-methoxyphenyl)sulphonyqamino] piperidine (1.54);
N-benzyl-4-[(N-3, 5-dimethoxyphenyl)-N-(4-methoxyphenyl)sulphonyl)amino]-piperidine (1.55);
N-benzyl-4-[(N-3-trifluoromethoxyphenyl)-N-(4-mothoxyphenylsulphonyl)amino]-piperidine (1.56);
N-benzyl-4-[(N-3-isopropylphenyl)-N-(4-methoxyphenyl)sulphonyl)amino]-piperidine (1.57);
N-benzyl-4-[(N-3-ethoxyphenyl)-N-(4-methoxyphenyl)sulphonyl)amino]piperidine (1.58);
N-benzyl-4-[(N-3-isopropoxyphenyl)-N-(4-methoxyphenyl)sulphonyluamino]-piperidine (1.59);
N-benzyl-4-[(N-3-phenoxyphenyl)-N-(4-methoxyphenyl)sulphonyl)amino] piperidine (1.60);
N-α-methylbenzyl-4-[(N-3-trifluoromethoxyphenyl)-N-(4-methoxyphenyl) sulphonyl)amino]-piperidine (1.61);
N-2-methylbenzyf-4-[(N-3-trifluoromethoxyphenyl)-N-(4-methoxyphenyl) sulphonyl)amino]-piperidine (1.62);
N-2-methoxylbenzyl-4-[(N-3-trifluoromethoxyphenyl)-N-(4-methoxyphenyl) sulphonyl)-amino]piperidine (1.63);
N-2-ethoxybenzyl-4-[(N-3-trifluoromethoxyphenyl)-N-(4-methoxyphenyl) sulphonyl)amino]-piperidine (1.64);
(R,S)-(trans)-N-Benzyl-3-methyl-4-[(N-phenyl-N-(4-methoxyphenyl)sulphonyl) amino]-piperidine (1.66);
(R, S)-(cis)-N-Benzyl-3-methyl-4-[(N-(3-methoxyphenyl)-N-(4-methoxyphenyl) sulphonyl)-amino]piperidine (1.67);
N-Benzyl-3-[(N-3-methoxyphenyl-N-4-methoxyphenylsulphonyl)amino] piperidine (1.70);
N-Benzyl-3-[(N-3-methoxyphenyt-N-phenylsulphonyl)aminomethyllpiperidine (1.71);
N-Benzyl-3-[(N-3-methoxyphenyl-N-4-methoxyphenylsulphonyl)aminomethyll piperidine(1 .72);
N-2-Cyanobenzyl-4-[(N-3-methoxyphenyl)-N-(4-methoxyphenyl)sulphonyl) amino]piperidine (1.73);
N-2-Nitrobenzyl-4-[(N-3-methoxyphenyl)-N-(4-methoxyphenyl)sulphonyl) amino]piperidine (1.74);
N-2-Fluorobenzyl4-[(N-3-methoxyphenyl)-N-(4-methoxyphenyl)sulphony1) amino]piperidine (1.75);
N-3-Fluorobenzyl-4-[(N-3-methoxyphenyl)-N-(4-methoxyphenyl)sulphonyl) amino]piperidine (1.76);
N-4-Fluorobenzyl-4-[(N-3-methoxyphenyl)-N-(4-methoxyphenyl)sulphonyl) amino]piperidine (1.77);
N-3, 5-Difluorobenzyl-4-[(N-3-methoxyphenyl)-N-(4-methoxyphenyl)sulphonyl) amino]piperidine (1.78);
N-2, 6-Difluorobenzyl-4-[(N-3-methoxyphenyl)-N-(4-methoxyphenyl)sulphonyl) amino]piperidine (1.79);
N-Cinnamyl-4-[(N-3-methoxyphenyl)-N-(4-methoxyphenyl)sulphonyl) amino]piperidine (1.80);
N-2-Phenylethyl-4-[(N-3-methoxyphenyl)-N-(4-methoxyphenyl)sulphonyl) amino]piperidine (1.81);
N-2-Pyridylmethyl-4-[(N-3-methoxyphenyl)-N-(4-methoxyphenyl)sulphonyl) amino]piperidine (1.82);
N-2-QuinoIylmethyl-4-[(N-3-methoxyphenyl)-N-(4-methoxyphenyl)sulphonyl) amino]piperidine (1.83);
N-3-Thienylmethyl-4-[(N-3-methoxyphenyl)-N-(4-methoxyphenyl)sulphonyl) amino]piperidine (1.85);
N-2-Furylmethyl-4-[(N-3-methoxyphenyl)-N-(4-methoxyphenyl)sulphonyl) amino]piperidine (1.85);
N-3-pyridylmethyl-4-[(N-3-methoxyphenyl)-N-(4-methoxyphenyl)sulphonyl) amino]piperidine (1.86);
N-2-ethoxybenzyl-4-[(N-3-methoxyphenyl)-N-(4-methoxyphenyl)sulphonyl) amino]piperidine (1.87);
N-2, 3-methylendioxybenzyl-4-[(N-3-methoxyphenyl)-N-(4-methoxyphenyl)sulphonyl)amino]piperidine (1.88);
N-2-Methoxybenzyl-4-[(N-3-methoxyphenyl-N-2, 5dimethoxy-4-nitrophenylsulphonyl)amino]piperidine (1.89);
N-benzyl-4-[N-(3-(trifluoromethoxy)phenyl)-N-(4-fluorophenylsulphonyl) amino]-piperidine (1.90);
N-benzyl-4-[N-(phenyl)-N-(3-methoxyphenylsulphonyl)amino]-piperidine (1.91);
N-benzyl-4-[N-(phenyl)-N-(4-(trifluoromethoxy)pheny1sulphonyl)amino]-piperidine (1.92);
N-benzyl-4-[N-(4-methylphenyl)-N-(3-methoxyphenylsulphonyl)amino]-piperidine (1.93);
N-benzyl-4-[N-(4-methylphenyl)-N-(4-(trifluoromethoxy)phenylsulphonyl) amino]-piperidine (1.94);
N-benzyl-4-[N-(4-methylphenyl)-N-(2-naphthylsulphonyl)amino]-piperidine (1.95);
N-benzyl-4-[N-(3-(trifluoromethoxy)phenyt)-N-(3-methoxyphenylsulphonyl) amino]-piperidine (1.96);
N-benzyl-4-[N-(3-trifluoromethoxy)phenyf)-N-(2-cyanophenylsufphonyl) amino]-piperidine (1.97);
N-benzyl-4-[N-(3-trifluoromethoxy)phenyl)-N-(3-cyanophenylsulphonyl) amino]-piperidine (1.98);
N-benzyl-4-[N-(3-trifluoromethoxy)phenyl)-N-(2-naphthylsulphonyl)amino]-piperidine (1.99);
N-(pytidin-4-ylmethyl)-4-[N-(3-trifluoromethoxy)phenyl)-N-(4-methoxyphenylsulphonyl)amino]-piperidine (1.100);
N-(2-chlorobenzyl)-4-[N-(3-trifluoromethoxy)phenyl)-N-(4-methoxyphenylsulphonyl)amino]-piperidine (1.101);
N-benzyl-4-[N-(3-trifluoromethoxy)phenyl)-N-(4-methylphenylsulphonyl) amino]-piperidine (1.102);
N-benzyl-4-[N-(3-trifluoromethoxy)phenyl]-N-(4-tert-butylphenylsulphony-) amino]-piperidine (1.103);
N-benzyl-4-[(4-t-butylphenyl-N-4-methoxyphenylsulphonyl)amino]piperidine (1.105);
N-benzyl-4-[(4-trifluoromethyl-N-4-methoxyphenylsulphonyl)amino] piperidine (1.106);
N-benzyl-4-[(3-trifluoromethyl-N-4- methoxyphenylsuiphonyl)amino] piperidine (1.107);
N-benzyl-4-[(4-propylphenyl-N-4- methoxyphenylsulphonyl)amino]piperidine (1.108);
N-benzyl-4[(N-3trifluoromethoxyphenyl-N-4-hydroxyphenylsulphonyl) amino]piperidine (1.109);

27. A compound selected from the group consisting of:
N-Benzoyl-4-[(N-benzyf-N-4-methoxyphenylsulphonyl)amino]-piperidine (1.1);
N-Phenyl-4-[(N-phenyl-N-4-methoxyphenylsulphonyl)amino]-piperidine (1.110);
N-Benzyl-4-[(N-2-methoxyphenyl-N-4-methoxyphenylsulphonyl)amino]-piperidine (1.2);
N-Benzyl-4-[(N-benzyl-N-4methoxyphenylsulphonyl)amino]-piperidine (1.3);
N-Benzyl-4-[(N-2-phenylethyl-N-(4-methoxyphenylsulphonyl)amino]-piperidine (1.4);
N-Benzyl-4-[(N-phenyl)-N-(4-methoxyphenyl)acetyl)amino]-piperidine (1.5);>
N-Benzyl-4-[(N-phenyl)-N-(phenoxy)acetyl)amino]-piperidine (1.6);
N-Benzyl-4-[(N-4-methoxybenzyl-N-4-methoxyphenylsulphonyl)amino;-piperidine (1.9);
N-Benzyl-4-[(N-phenyl)-N-((2-benzo[b]thiophenylucarbonyl)amino]-piperidine (1.7);
N-Benzyl-4-[(N-phenyl-N-4-methoxyphenylsulphonyl)amino]-piperidine (1.8);
N-Benzyl-4-[(N-4-methoxybenzyl-N-4-chlarophenylsulphonyl)amino]-piperidine (1.11);
N-Benzyl-4-[(N-2-phenylethyl-N-phenylsulphonyl)amino]-piperidine (1.15);
N-Benzyl-4-[(N-2-phenylethyl-N-(4-chlorophenyl)sulphonyl)amino]-piperidine (1.16);
N-Benzyl-4-[(N-2-phenylethyl-N-(4-methylphenyl)sulphonyl)amino]-piperidine (1.17);
N-Benzyl-4-[(N-2-phenylethyl-N-(2,5-dimethoxyphenyl)sulphonyl)amino]-piperidine (1.19);
N-Benzyl-4-[(N-phenyl-N-(4-chlorophenyl)sulphonyl)amino]-piperidine (1.21);
N-Benzyl-4-[(N-phenyl-N-(4-methylphenyl)sulphonyl)amino]-piperidine (1.22);
N-((2-fu(yl)methyl)-4-[(N-benzyl-N-4-methoxyphenylsulphonylwamino]-piperidine (1.112);
N-Benzyl-4-[(N-(2-naphthyl)methyl)-N-(4-methoxypheny)sulphonyl)amino-piperidine (1.28);
N-Benzyl-4-[(N-1-naphthylmethyl)-N-(4-methoxyphenyl)sulphonyl)amino]-piperidine (1.29);
N-Benzyl-4-[(N-(4-methoxyphenyl)-N-(4-methoxyphenyl)sulphonyluamino]-piperidine (1.30);
N-Benzyl-4-[(N-4-chlorophenyl)-N-(4-methoxyphenyl)sulphonyl)amino]-piperidine (1.31);
N-(2phonethyl)-4-[(N-phenyl-N-(4-methoxyphenyl)sulphonyl)amino]-piperidine (1.111);
N-Benzyl-4-[(N-(2-naphthyl)-N-(4-methoxyphenylusulphonyl)amino]-piperidine (1.33);
N-Benzyl-4-[(N-3-methoxyphenyl)-N-(4-methoxyphenyl)sulphonyl)amino]-piperidine (1.34);
N-Benzyl-4-[(N-3-chlorophenyl)-N-(4-methoxyphenyl)sulphonyl)amino]-piperidine (1.35);
N-Benzyl-4-[(N-4-methylphenyl)-N-(4-methoxyphenyl)sulphonyl)amino]-piperidine (1.36);
N-Benzyl-4-[(N-4-isopropylphenyl)-N-(4-methoxyphenyl)sulphonyf)amino]-piperidine (1.37);
N-Benzyl-4-[(N-4-trifruoromethoxyphenyl)-N-(4-methoxyphenyl)sulphonyl)amino]-piperidine (1.38);
N-Benzyl-4-[(N-3-mothylphenyl)-N-(4-methoxyphenyl)sulphonyl)amino]-piperidine (1.40);
N-Benzyl-4-[(N-15-indanyl))-N-(4-methoxyphenyl)sulphanyl;amino]-piperidine (1.41);
N-Benzyl-4-[(N-3,4-dichlorophenyl)-N-(4-methoxyphenyl)sulphonyl)amino]-piperidine (1.42);
N-Benzyl-4-[(N-3-chloro-4-methoxyphenyl)-N-(4-methoxyphenyl)sulphonyl)amino]-piperidine (1.43);
N-Benzyl-4-[(N-3,4-methylendioxyphenyl)-N-(4-methoxyphenyl)sulphonyl)amino]-piperidine (1.44);
N-(2-furylmethyl)-4-[(N-3-methoxyphenyl)-N-(4-methoxyphenyl)sulphonyl)amino]-piperidine (1.45);
N-3-chlorobenzyl-4-[(N-3-methoxyphenyl)-N-(4-methoxyphenyl)sulphonyI)amino]-piperidine (1.46);
N-(2-Chlorobenzyl)-4-[(N-3-methoxybenzyl)-N-(4-methoxyphenyI)sulphonyl)amino]-piperidine (1.47);
N-2-methylbenzyl-4-[(N-3-methoxyphenyl)-N-(4-methoxyphenyl)sulphonyl)amino]-piperidine (1.48);
N-2-methoxybenzyl-4-[(N-3-methoxyphenyl)-N-(4-methoxyphenyl)sulphonyl)amino]-piperidine (1.49);
N-3-methoxybenzyl-4-[(N-3-methoxyphenyl)-N-(4-methoxyphenyl)sulphonyl)amino]-piperidine (1.50);
N-4-methoxybenzyl-4-[(N-3-methoxyphenyl)-N-(4-methoxyphenyl)sulphonyl)amino]-piperidine (1.51);
N-3-methylbenzyl-4-[(N-3-methoxyphonyl)-N-(4-mothoxyphenyl)sulphonyl)amino]-piperidine (1.52);
N-4-methylbenzyl-4-[(N-3-methoxyphenyl)-N-(4-methoxyphenyl)sulphonyl)amino]-piperidine (1.53);
N-4-chlorobenzyl-4-[(N-3-methoxyphenyl)-N-(4-methoxyphenyl)sulphonylpamino]-piperidine (1.54);
N-benzyl-4-[(N-3, 5-dimethoxyphenyl)-N-(4-methoxyphenyl)sulphonyl)amino]-piperidine (1.55);
N-benzyl-4-[(N-3-trifluoromethoxyphenyl)-N-(4-methoxyphenyl)sulphonyl)amino]-piperidine (1.56);
N-benzyl-4-[(N-3-isopropyfphenyl)-N-(4-methoxyphenyl)sulphonyl)amino]-piperidine (1.57);
N-benzyl-4-[(N-3-ethoxyphenyl)-N-(4-methoxyphenyl)sulphonylvamino]-pipeiidine (1.58);
N-benzyl-4-[(N-3-isopropoxyphenyl)-N-(4-methoxyphenylpsulphonylpamino]-piperidine (1.59);
N-α-methylbenzyl-4-[(N-3-trifluoromethoxyphenyl)-N-(4-methoxyphenyl) sulphonyl)amino]-piperidine (1.61);
N-2-methylbenzyl-4-[(N-3-trifluoromethoxyphenyl)-N-(4-methoxyphenyl) sulphonyl)amino]-piperidine (1.62);
N-2-methoxylbenzyl-4-[(N-3-trifluoromethoxyphenyl)-N-(4-methoxyphenyl) sulphonyl)-amino]piperidine (1.63);
N-2-ethoxybenzyf-4-[(N-3-trifluoromethoxyphenyl)-N-(4-methoxyphenyl) sulphonyl)amino]-piperidine (1.64);
(R,S)-(trans)-N-Benzyl-3-methyl-4-[(N-phenyl-N-(4-methoxyphenyl)sulphonyl) amino]-piperidine (1.66);
(R,S)-(cis)-N-Benzyl-3-methyl-4-[(N-(3-methoxyphenyl)-N-(4-methoxyphenyl) sulphonyl)-amino]piperidine (1.67);
N-Benzyl-3-[(N-3-methoxyphenyl-N-4-methoxyphenylsulphonyl)amino] piperidine (1.70);
N-Benzyl-3-[(N-3-methoxyphenyl-N-phenylsulphonyl)aminomethy]piperidine (1.71);
N-2-Cyanobenzyl-4-[(N-3-methoxyphenyl)-N-(4-methoxyphenyl)sulphonyl) amino]piperidine (1.73);
N-2-Nitrobenzyl-4-[(N-3-methoxyphenyl)-N-(4-methoxyphenyl)sulphonyl) amino]piperidine (1.74);
N-2-Fluorobenzyl-4-[(N-3-methoxyphenyl)-N-(4-methoxyphenyl)sulphonyl) amino]piperidine (1.75);
N-3-Fluorobenzyl-4-[(N-3-methoxyphenyl)-N-(4-methoxyphenyl)sulphonyl) amino]piperidine (1.76);
N-4-Fluorobenzyl-4-[(N-3-methoxyphenyl)-N-(4-methoxyphenyl)sulphonyl) amino]piperidine (1.77);
N-3, 5-Difluorobenzyl-4-[(N-3-methoxyphenyl)-N-(4-methoxyphenyl)sulphonyl) amino]piperidine (1.78);
N-2, 6-Difluorobenzyl-4-[(N-3-methoxyphenyl)-N-(4-methoxyphenyl)sulphonyl) amino]piperidine (1.79);
N-Cinnamyl-4-[(N-3-methoxyphenyl)-N-(4-methoxyphenyl)sulphony1) amino]piperidine (1.80);
N-2-Phenylethyl-4-[(N-3-methoxyphenyl)-N-(4-methoxyphenyl)sulphonyl) amino]piperidine (1.81);
N-2-Pyridyfmethyl-4-[(N-3-methoxyphenyl)-N-(4-methoxyphenyl)sulphonyl) amino]piperidine (1.82);
N-2-Quinolylmethyl-4-[(N-3-methoxyphenyl)-N-(4-methoxyphenyl)sulphonyl) amino]piperidine (1.83);
N-3-Thienylmethyl-4-[(N-3-methoxyphenyl)-N-(4-methoxyphenyl)sulphonyl) amino]piperidine (1.85);
N-2-Furylmethyl-4-[(N-3-methoxyphenyl)-N-(4-methoxyphenyl)sulphonyl) amino]piperidine (1.85);
N-3-pyridylmethyl-4-[(N-3-methoxyphenyl)-N-(4-methoxyphenyl)sulphonyl) amino]piperidine (1.86);
N-2-ethoxybenzyl-4-[(N-3-methoxyphenyt)-N-(4-methoxyphenyl)sulphonyl) amino]piperidine (1.87);
N-2,3-methylendioxybenzyl-4-[(N-3-methoxyphenyl)-N-(4-methoxyphenyl)sulphonyl)amino]piperidine (1.88);
N-2-Methoxybenzyl-4-[(N-3-methoxyphenyl-N-2, 5-dimethoxy-4-nitrophenylsulphonyl)amino]piperidine (1.89);
N-benzyl-4-[N-(3-(trifluoromethoxy)phenyl)-N-(4-fluorophenylsulphonyl) amino]-piperidine (1.90);
N-benzyl-4-[N-(phenyl)-N-(3-methoxyphenylsulphonyl)amino]-piperidine (1.91);
N-benzyl-4-[N-(phenyl)-N-(4-(trifluoromethoxy);phenysulphonyI)amino]-piperidine (1.92);
N-benzyl-4-[N-(4-methylph1nyl)-N-(3-methoxyphenylsulphonyl)amino-piperidine (1.93);
N-benzyl-4-[N-(4-methylphenyl)-N-(4-(trifluoromethoxy)phenylsulphonyl) amino]-piperidine (1.94);
N-benzyl-4-[N-(4-methylphenyl)-N-(2-naphthylsulphonyl)amino]-piperidine (1.95);
N-benzyl-4-[N-(3-(trifluoromethoxy)phenyl)-N-(3-methoxyphenylsulphonyl) amino]-piperidine (1.96);
N-benzyl-4-[N-(3-trifluoromethoxy)phenyl)-N-(2-cyanophenylsulphonyl) amino]-piperidine (1.97);
N-benzyl-4-[N-(3-trifluoromethoxy)phenyl)-N-(3-cyanophenylsulphonyl) amino]-piperidine (1.98);
N-benzyl4-[N-(3-trifluoromethoxy)phenyl)-N-(2-naphthyisulphonyl)amino]-piperidine (1.99);
N-(pyridin-4-ylmethyl)-4-[N-(3-trifluoromethoxy)phenyl)-N-(4-methoxyphenylsulphonyl)amino]-piperidine (1.100);
N-(2-chlorobenzyl)-4-[N-(3-trifluoromethoxy)phenyl)-N-(4-methoxyphenylsulphonyl)amino]-piperidine (1.101);
N-benzyl-4-[N-(3-trifluoromethoxy)phenyl)-N-(4-methylphenylsulphonyl) amino]-pipendine (1.102);
N-benzyl-4-[N-(3-trifluoromethoxy)phenyl)-N-(4-tert-butylphenylsulphony1) amino]-piperidine (1.103);
N-benzyi-4-[(4-t-butylphenyl-N-4-methox henylsulphonyl)amino]piperidine (1.105);
N-benzyl-4-[(4-trifluoromethyl-N-4-methoxyphenylsulphonyl)amino]pipetidine (1.106);
N-benzyl-4-[(3-trifluoromethyl-N-4- methoxyphenylsulphonyl)amino] piperidine (1.107);
N-benzyl-4-[(4-propylphenyl-N-4- methoxyphenylsulphonyl)amino] piperidine (1. 108);
N-benzyl-4[(N-3trifluoromethoxyphenyl-N-4-hydroxyphenylsulphonyl) amino]piperidine (1.109);

28. A composition comprising a compound of claim 1 and a carrier.

29. A pharmaceutical composition comprising a therapeutically effective amount of a compound of claim 1 and pharmaceutically acceptable carrier.

30. A method for treating a patient having a medical condition for which a glycine transport inhibitor is indicated, comprising the step of administering to the patient a pharmaceutical composition as defined in claim 29.

31. A method according to claim 30 in which the medical condition is schizophrenia.

32. A method according to claim 30 in which the medical condition is cognitive dysfunction.

33. A method according to claim 30 in which the medical condition is Alzheimer's disease.
